(19) United States
(12) Patent Application Publication
(10) Pub. No.: US 2025/0101383 A1
(43) Pub. Date: Mar. 27, 2025

(54) METHODS AND COMPOSITIONS FOR CARDIAC MODELS

(71) Applicant: Sanford Burnham Prebys Medical Discovery Institute, La Jolla, CA (US)

(72) Inventors: Alexandre Romain COLAS, La Jolla, CA (US); Anaïs KERVADEC-BADILLO, San Diego, CA (US); Michael Shenghan YU, Cambridge, MA (US)

(21) Appl. No.: 18/889,804

(22) Filed: Sep. 19, 2024

Related U.S. Application Data
(60) Provisional application No. 63/584,370, filed on Sep. 21, 2023.

(51) Int. Cl. C12N 5/077; C12N 15/113

(52) U.S. Cl. CPC C12N 5/0657; C12N 15/113; C12N 2310/14; C12N 2502/1323; C12N 2503/02; C12N 2506/45; C12N 2510/00

(57) ABSTRACT

The present disclosure provides for in-vitro generated cardiomyocytes, as well as methods of using such cardiomyocytes or variants thereof. The present disclosure also relates to methods of cell co-culture models of cardiac disorders, as well as methods of using such models or variants thereof.

### CROSS REFERENCE TO RELATED APPLICATION

Any and all priority claims identified in the Application Data sheet, or any correction thereto, are hereby incorporated by reference under 37 CFR 1.57. For example, this Application claims the benefit of U.S. Provisional App. No. 63/584,370 filed on Sep. 21, 2023, which is incorporated by reference in its entirety herein.

### STATEMENT AS TO FEDERALLY SPONSORED RESEARCH

This invention was made with government support under R01 HL153645, R01 HL148827, R01 HL149992, and R01 AG071464 awarded by the National Institutes of Health. The government has certain rights in the invention.

### BACKGROUND

Atrial fibrillation (AF) is a common form of sustained cardiac arrhythmia in humans (Du et al., 2017). At the whole heart level, a central feature of AF is a very rapid and uncoordinated atrial activity while at the cellular level, the mechanism maintaining arrhythmia often arises from a “vulnerable substrate”, which consists of either action potential duration (APD) prolongation or shortening events (Christophersen et al., 2013). Such vulnerable substrates have been linked to genetic predispositions, cardiac remodeling caused by heart disease, aging, and/or altered regulation by neurohormonal factors. linkage analysis in familial cases of AF as well as genome-wide associated studies (GWAS) in the general population have elucidated some of the genetic underpinnings associated with the disease with close to 140 genetic loci linked to >200 genes have been identified, however none of these genes have been validated as disease-causing in the general population, limiting drug discovery efforts. In this context, a major barrier to progress is the lack of experimental platforms/strategies enabling rapidly establishment of causal links between gene function and AF-associated phenotypes (electrical remodeling, arrhythmia).

The four-chambered mouse heart has been used to establish functional links between genes or genetic loci and rhythm phenotypes (Lozano-Velasco et al., 2016; Nadadur et al., 2016; Temple et al., 2005; van Ouwerkerk et al., 2019; Wang et al., 2010; Zhang et al., 2019). However, despite proteome homology with humans and ability to manipulate the genome, the substantial electrophysiological differences (fast resting rate, short AP duration and triangular shape, species-specific K+ channels (Kaese and Verheule, 2012)), relatively long lifespan (years) and low throughput capacity of methods to retrieve electrophysiological parameters, limit the use of mice as a primary model for gene discovery related to AF. In contrast to mice, flies have a short generation time (˜10 days) and established automated kinetic imaging techniques (Fink et al., 2009; Klassen et al., 2017), coupled with available functional genomic resources (e.g. Flybase.org; VDRC (Mohr et al., 2014)), enabling the rapid evaluation of gene function on rhythm parameters at the whole heart level, although a limitation to this model is the lack of atrial specificity.

Although human iPSC-derived atrial-like cardiomyocytes (ACMs) can be used identify atrial-specific and cell autonomous rhythm-regulating mechanisms, the relative immaturity of hiPSCs-derived CMs and inherent lack of tissue level integration, might limit translation of the findings to the adult human heart. In sum, single model approaches are limited in their ability to validate large cohorts of AF-associated genes, indicating the necessity to develop alternative strategies to improve AF gene validation.

### SUMMARY

Combining assays with human, atrial, and whole organ relevance that also have HT functional genomics capacity could enhance our ability to rapidly establish causal links between AF-associated genes and arrhythmia phenotypes. The disclosure provided herein established such a platform. A human-relevant assay was developed that measures APD in ACMs with single cell resolution. In parallel, a fly cardiac function assay was optimized that measures contraction duration (systolic interval (SI)), as a surrogate measurement for APD. A cohort of 20 AF-associated genes was screened, and Phospholamban (PLN) loss of function was identified as a conserved gene that surprisingly and significantly shortens action potential duration in ACMs, HAMs and fly cardiomyocytes. Remarkably, addition of environmental stressors (i.e fibroblasts, β-adrenergic stimulation), further increased the generation of irregular beat to beat intervals, delayed after depolarizations, and triggered action potentials, in PLN knockdown cells as compared to controls. To delineate the mechanism underlying PLN KD-dependent arrhythmia, a logistic regression approach was used in HAM populations which predicted that PLN functionally interacts with both NCX (loss of function) and L-type calcium channels (gain of function) to mediate these arrhythmic phenotypes. Co-KD of PLN and NCX in ACMs and flies led to increased arrhythmic events, while treatment of ACMs with L-type calcium channel inhibitor, verapamil, reverted these phenotypes. The platform described herein provides in-depth resolution of cardiac electrophysiology metrics that can be used in various applications such as, for example, (1) performing large-scale functional genomic screens to identify novel gene regulatory networks governing cardiac rhythm, (2) creating new arrhythmia models to phenotypically characterize rhythm-associated cardiac diseases and stressors that affect the disease; (3) screening small-molecules to discovery new anti-arrhythmic therapeutics; and (4) determining the effects of the cardio microenvironment (e.g. by co-culturing with fibroblasts) on rhythm-associated cardiac diseases.

In an aspect, the present disclosure describes an in vitro-generated cardiomyocyte. In some embodiments, the in vitro-generated cardiomyocyte is generated from a reprogrammed cell in vitro. In some embodiments, the in vitro-generated cardiomyocyte comprises at least one gene associated with a cardiac rhythm disorder having an altered expression status. In some embodiments, the in vitro-generated cardiomyocyte displays a phenotype associated with the cardiac rhythm disorder. In some embodiments, the cardiac rhythm disorder is atrial fibrillation (AF). In some embodiments, the cardiomyocyte is an atrial-like cardiomyocyte (ACM). In some embodiments, the gene associated with the cardiac rhythm disorder is selected from a group comprising GATA5, GATA6, PITX2, KCNA5, GATA4, KCNJ5, HCN4, GJA1, TBX5, SYNE2, NKX2-6, SH3PXD2A, KCNN3, NPPA, ZFHX3, NKX2-5, HAND2, GJA5, KCND3, and PLN.

In some embodiments, the phenotype associated with the cardiac rhythm disorder is an alteration in a cardiac rhythm parameter. In some embodiments, the cardiac rhythm parameter may be selected from a group comprising action potential duration (APD), systolic interval, beat rate, beat refractory period, peak-to-peak interval, early afterdepolarization, delayed afterdepolarization, and Arrythmia Index (AI) value. In some embodiments, the phenotype is an AI value greater than 20. In some embodiments, the alteration in the cardiac rhythm parameter is a change in the APD75 value, wherein the APD75 value is APD measured at 75% repolarization. In some embodiments, the alteration in the cardiac rhythm parameter is a change in the APD90 value, wherein APD90 is APD measured at 90% repolarization. In some embodiments, the alteration in the cardiac rhythm parameter is a shortening of the APD as compared to a reference cardiomyocyte. In some embodiments, the alteration in the cardiac rhythm parameter is an increase in the beat refractory period as compared to a reference cardiomyocyte. In some embodiments, the alteration in the cardiac rhythm parameter is an increase in beat rate as compared to a reference cardiomyocyte. In some embodiments, the alteration in the cardiac rhythm parameter is a reduction in the systolic interval as compared to a reference cardiomyocyte. In some embodiments, the alteration in the cardiac rhythm parameter is a shortening of the Ca2+ transient duration as compared to a reference cardiomyocyte. In some embodiments, the altered expression status is overexpression of the at least one gene associated with the cardiac rhythm disorder as compared to the expression level of the gene in a reference cardiomyocyte. In some embodiments, the altered expression status is reduced expression of the at least one gene associated with the cardiac rhythm disorder as compared to the expression level in a reference cardiomyocyte.

In some embodiments, the in vitro-generated cardiomyocyte further comprises a nucleic acid molecule capable of modulating the expression of the at least one gene associated with the cardiac rhythm disorder. In some embodiments, the nucleic acid molecule is siRNA.

In some embodiments, the reprogrammed cell is a cardiac progenitor cell. In some embodiments, the cardiac progenitor cell overexpresses Id1. In some embodiments, the reprogrammed cell is an induced pluripotent stem cell (iPSC).

In some embodiments, the cardiomyocyte expresses one or more genes selected from a group comprising NR2F2, TBX5, ZNF385B, KCNJ3, KCNA5, NPPA, NPPB, EGR1/2 and PDGFRA.

In an aspect, the present disclosure describes a cell population comprising at least two in vitro-generated cardiomyocytes of the present disclosure. In some embodiments, the percentage of the cell population that exhibits an AI value of at least 20 is greater than 20%, 30%, 40%, 50%, 60%, 70% or 80%. In some embodiments, the APD75 value measured using a Kolmogorov-Smirnov scale is at least 0.2, 0.3, 0.4, 0.5, 0.6, 0.7, 0.8, or 0.9 when compared to the APD75 value of a reference cardiomyocyte cell population.

In an aspect, the present disclosure describes a cell co-culture model of cardiac fibrosis comprising the in vitro-generated cardiomyocyte of the present disclosure and a fibroblast cell. In some embodiments, the ratio of the fibroblast cell to the cardiomyocyte cell is 3:1.

In an aspect, the present disclosure describes a cell co-culture model of cardiac arrhythmia comprising the in vitro-generated cardiomyocyte of the present disclosure and a pharmaceutical compound. In some embodiments, the pharmaceutical compound is isoproterenol. In some embodiments, the pharmaceutical compound is dofetilide.

In an aspect, the present disclosure provides a method for screening a candidate agent for the treatment of a cardiac rhythm disorder comprising contacting the cardiomyocyte of the present disclosure with the candidate agent and detecting an effect of the candidate agent on the phenotype associated with the cardiac rhythm disorder. In some embodiments, the method further comprises culturing the cardiomyocyte with a fibroblast cell prior to contacting with the candidate agent. In some embodiments, the method further comprises labeling the cardiomyocyte with a voltage dye or a nuclear dye. In some embodiments, the method further comprises quantifying the voltage-dependent fluorescence variation over time. In some embodiments, the method further comprises automatically processing the action potential trace. In some embodiments, the method further comprises measuring parameters selected from the group comprising: APD-10, 25, 50, 75, 90; T25-75, T75-25; Vmax up and down; beat rate; peak-to-peak interval; and rhythm regularity index. In some embodiments, the candidate agent is a nucleic acid. In some embodiments, the candidate agent is a small molecule. In some embodiments, the candidate agent is a protein. In some embodiments, the nucleic acid recognizes the gene phospholamban (PLN). In some embodiments, the nucleic acid recognizes the gene NCX. In some embodiments, the candidate agent is isoproterenol. In some embodiments, the candidate agent is dofetilide. In some embodiments, the candidate agent is verapamil. In some embodiments, the detecting is conducted at single cell resolution.

In an aspect, the present disclosure provides a method of determining an increased risk for atrial fibrillation (AF) in a human subject comprising collecting a biological sample from the human subject and determining by an assay a level of a gene or gene product associated with AF in the biological sample. In some embodiments, the assay further comprises contacting the biological sample with a reagent that recognizes the gene or gene product associated with AF. In some embodiments, the biological sample is blood from the human subject. In some embodiments, the biological sample is a DNA sample. In some embodiments, the assay comprises genome sequencing of the human subject. In some embodiments, the assay comprises a proteomic assay. In some embodiments, the method further comprises administering a therapeutic agent to the human subject, wherein the therapeutic agent is configured to mitigate or alleviate one or more symptoms of AF in the human subject. In some embodiments, the gene is phospholamban (PLN). In some embodiments, the gene is NCX. In some embodiments, the gene product is a L-type Calcium channel. In some embodiments, the therapeutic agent is verapamil.

In an aspect, the present disclosure provides a method for high-throughput identification of a gene underlying a cardiac rhythm disorder comprising evaluating the effect of the loss-of-function and gain-of-function of the gene on the in vitro-generated cardiomyocyte of the present disclosure, evaluating the effect of the loss-of-function and gain-of-function of the gene on a Drosophila heart, or computational modeling of the effect of knockdown of the gene on a computational model of heterogenous adult human atrial myocytes (HAMs). In some embodiments, the cardiac rhythm disorder is AF. In some embodiments, the evaluating comprises measuring a phenotype associated with the cardiac rhythm disorder, wherein the phenotype is an alteration in a cardiac rhythm parameter. In some embodiments, the cardiac rhythm parameter is selected from a group comprising action potential duration (APD), systolic interval, beat rate, beat refractory period, peak-to-peak interval, early afterdepolarization, delayed afterdepolarization, and Arrythmia Index (AI) value.

In some embodiments, the evaluating comprises measuring parameters of Drosophila heart function selected from the group comprising: heart period (R-R interval), systolic interval (SI), arrhythmicity, fractional shortening, and contractility. In some embodiments, the modeling further comprises simulating parameters selected from a group comprising: affinity of the sarco/endoplasmic reticulum Ca2+-ATPase (SERCA) for cytosolic Ca2+, maximum ion channel conductances, rates for membrane transporters, and Ca+2 handling fluxes. In some embodiments, the method further comprises simulating the parameters on a cell population basis.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Additionally, this application is related to the following Disease Models & Mechanisms research article: Kervadec A, et al. Multiplatform modeling of atrial fibrillation identifies phospholamban as a central regulator of cardiac rhythm. Dis Model Mech. 2023 Jul. 1; 16 (7):dmm049962. doi: 10.1242/dmm.049962. Epub 2023 Jul. 17, which became available on Jul. 17, 2023, and which is incorporated herein by reference in its entirety.

### BRIEF DESCRIPTION OF THE DRAWINGS

FIG. 1 shows the generation of atrial-like cardiomyocytes (ACMs) from cardiac progenitor cells.

FIG. 2A shows a graph quantifying the percentage of NR2F2 and ACTN2 positive cells among ACMs and ventricular cardiomyocytes (VCMs). FIG. 2B shows immunofluorescence images of ACMs and VCMs stained for ACTN2 (green) and NRF2 (red) expression. The inlaid image at the top right corner of each ACTN2 image shows the same cells stained with DAPI.

FIG. 2C shows a heatmap of atrial genes enriched in day 12 ACMs as compared to VCMs.

FIG. 3 shows a representative triangular-shaped action potential recorded in a day 25 ACM.

FIGS. 4A-D shows a schematic representation of the single cell and high throughput (HT) platform to measure action potential duration (APD) parameters in ACMs of the present disclosure. FIG. 4A shows a schematic of high throughput (HT) voltage kinetics imaging of ACMs. FIG. 4B shows a schematic of automated single cell segmentation and fluorescence quantification of the ACM model of the present disclosure. FIG. 4C shows a schematic and example graphs showing automated single-cell trace analysis using a custom analysis software cloud computing program of the present disclosure. FIG. 4D shows an example graph showing the phenotypic assessment of electrical remodeling, namely APD shortening (left arrow) or prolongation (right arrow).

FIG. 5A shows a population distribution graph of APD₇₅ values from ACMs treated with escalating doses of isoproterenol, which show a dose dependent APD shortening. FIG. 5B shows single action potential (AP) traces of median APD₇₅ for each condition. FIG. 5C shows a histogram quantifying the Kolmogorov-Smirnov distance values (KS-D) for control (Ctrl) and isoproterenol treated ACMs.

FIG. 6A and FIG. 6B show heatmaps of atrial genes enriched in day 25 ACMs as compared to VCMs.

FIG. 7 shows a representative AP trace with ventricular characteristics, including a long phase 2, collected using a patch-clamp experiment in a day 25 VCM.

FIG. 8A shows a histogram quantifying the average calcium transient duration (CTD₅₀) in ACMs vs VCMs. FIG. 8B shows a histogram quantifying the average CTD₇₅ in ACMs vs VCMs. FIG. 8C shows a representative calcium trace recorded in an ACM at day 25. FIG. 8D shows a representative calcium trace recorded in a VCM at day 25.

FIG. 9 shows a histogram with Arrhythmia Index (AI) values showing that values below 20 are classified as regular or not arrhythmic while values greater than 20 are classified as irregular or arrhythmic.

FIG. 10A shows example peak trains from ACMs with a regular AI phenotype. FIG. 10B shows example peak trains from ACMs with a generally regular AI phenotype. FIG. 10C shows example peak trains from ACMs with a mildly irregular AI phenotype. FIG. 10D shows example peak trains from ACMs with a severely irregular AI phenotype. FIG. 10E shows average AI values for each example phenotype shown.

FIG. 11A shows a histogram representing the distribution of AI values for ACMs treated with increasing doses of Dofetilide. FIG. 11B shows representative peak trains of control ACMs. FIG. 11C shows representative peak trains of ACMs treated with 33 nM Dofetilide. FIG. 11D shows representative peak trains of ACMs treated with 100 nM Dofetilide. FIG. 11E shows a histogram representing the proportion of arrhythmic cells with an AI value of greater than 20 in control ACMs and ACMs treated with 33 nM and 100 nM Dofetilide. FIG. 11F shows histograms quantifying KS-D values between control or Dofetilide-treated ACMs.

FIG. 12A shows a schematic of the fly thorax and abdomen (heart tube shown highlighted in gray in the center of the diagram above the 10× objective). FIG. 12B shows images of a semi-intact preparation (left) with a single cardiac chamber (inside the gray box and in the magnified image to the right).

FIG. 13A shows simultaneous optical and electrophysiological recordings from beating hearts. M-modes from optical recordings are shown on the top with the corresponding action potential (AP) traces below. FIG. 13B shows simultaneous optical and electrophysiological recordings from beating hearts. M-modes from optical recordings are shown on the top with the corresponding action potential (AP) traces below. The lower window shows the voltage trace generated by the image capture software that was used to synchronize the optical and electrical recordings. AP duration (APD) and systolic intervals (SI) are shown in seconds. FIG. 13C shows systolic intervals (SI) paired with corresponding APs. FIG. 13D shows the Pearson Correlation Coefficient for the combined data in FIG. 13C and shows a significant correlation between SIs and APDs (r=0.96, p<0.0001).

FIG. 14 shows a histogram representing the expression level (RPKM) of previously known AF-associated genes using RNA sequencing (RNAseq) data from Day 12 and Day 25 ACMs.

FIG. 15A shows a histogram showing the distribution of calcium transient duration (CTD) values in siControl and siPLN condition in ACMs (right) and representative single calcium transients for both conditions (left). FIG. 15B shows a histogram of CTD generated from HAMs (left) and representative traces (right) in response to Kmf25% condition (=PLN KD). P-value ***<0.0001.

FIG. 16 shows a heatmap showing the normalized effects of AF-associated genes loss of function on APD and SI in ACMs and flies, respectively.

FIG. 17A shows a population distribution histogram showing APD₇₅ values for siControl and siPLN transfected ACMs. FIG. 17B shows representative AP traces showing the shortening effect of siPLN. FIG. 17C shows representative m-modes showing the SI shortening effect for SclA KD. FIG. 17D shows representative AP traces (right) showing the shortening effect on APD of simulated PLN KD. FIG. 17E shows a population distribution histogram showing SIs in control vs SclA KD conditions in flies. FIG. 17F shows a population distribution histogram showing APD₉₀ values for Control and Kmf 25% (=PLN KD) in HAMs.

FIG. 18 shows a schematic representation of APD modeling in HAMs.

FIG. 19A shows a histogram showing the quantification of PLN staining intensity in siControl and siPLN conditions in ACMs. FIG. 19B shows representative images of ACMs stained for actinin2 (ACTN2, green, upper left inset boxes) to mark ACMs and for phospholamban (PLN, red, main panels) showing a significant reduction of PLN protein levels upon PLN KD. DAPI stain for each of the main panels is shown in the smaller inset boxes in the upper right corners.

FIG. 20A shows a histogram showing the distribution of AI values from ACMs in siControl and siPLN conditions. FIG. 20B shows representative AP traces in siControl and siPLN conditions. FIG. 20C shows the quantification of irregular AP peak trains in siControl and siPLN conditions.

FIG. 21 shows representative images of ACMs co-cultured with human dermal fibroblasts and stained with ACTN2 (cardiac, green, top) and TAGLN (fibroblasts, red, middle). DAPI nuclear stain (blue) is shown in all panels.

FIG. 22A shows a histogram showing the distribution of AI values from ACMs co-cultured with fibroblasts (Fib) in siControl and siPLN conditions. FIG. 22B shows quantification of the percentage of ACMs with irregular AP peak trains for each condition. FIG. 22C shows representative AP peak trains for each condition.

FIG. 23A shows a histogram showing the distribution of AI values from ACMs treated with Isoproterenol in siControl and siPLN conditions. FIG. 23B shows quantification of the percentage of ACMs with irregular AP peak trains for each condition. FIG. 23C shows representative AP peak trains for each condition.

FIG. 24A shows a histogram of arrhythmia index (AI) values of ACMs co-cultured with fibroblasts and treated with isoproterenol (Isop) in siControl vs siPLN conditions. FIG. 24B shows a histogram showing the increased percentage of irregularly beating (AI>20) ACMs co-cultured with fibroblasts and treated with Isop, in siPLN as compared to siControl. FIG. 24C shows representative peak trains of APs show irregular beat to beat interval (black arrowheads) in siPLN as compared to siControl condition.

FIG. 25A shows the distribution of Median Absolute Deviation (MAD) values before (left data points), during (center data points), and after 100 nM octopamine treatment (OA) (right data points). Post-OA, SclA KD hearts exhibit increased arrhythmia as compared to controls (p-value <0.05, repeated measures 2-way ANOVA). FIG. 25B shows representative M-modes showing irregular beat to beat intervals in SclA KD hearts post-OA as compared to control (arrows show individual heart periods).

FIG. 26A shows a graph showing average SI values in response to escalating doses of OA in flies. FIG. 26B shows representative M-modes from one heart before (top) and during application of 100 nM OA (bottom) showing dramatic increases in heart rate. FIG. 26C shows histograms showing the distribution of SI values in control hearts pre-OA pacing, during exposure to 100 nM OA, and 15 min post-OA application. Distribution of SIs post-OA returns to that of pre-OA. FIG. 26D shows histograms showing the distribution of SI values in hearts treated with SclA siRNA pre-OA pacing, during exposure to 100 nM OA, and 15 min post-OA application. Distribution of SIs post-OA are significantly shorter compared to pre-OA and are due to decreases in both the contraction and relaxation phases of the systolic intervals (p-value <0.05, repeated measures 2-way ANOVA). FIG. 26E shows graphs showing contraction intervals corresponding to the data shown in FIGS. 26C-D. FIG. 26F shows graphs showing relaxation intervals corresponding to the data shown in FIGS. 26C-D.

FIGS. 27A-C shows the modeling framework for evaluating arrhythmic events in HAMs and two-dimensional (2D) human atrial tissue. FIG. 27A shows a schematic depicting action potential modeling in human adult atrial myocytes (HAMs) on the left and an example graph of action potentials generated using the HAMs of the present disclosure on the right. FIG. 27B shows many differently shaded squares, each row corresponding to a different model, and each shade representing different electrophysiological properties and values. FIG. 27C shows triggered activity in response to a pacing-pause protocol.

FIGS. 28A-E shows the effects of PLN kd (Kmf 25%) and Isop on the triggered activity in human atrial cardiomyocytes. FIGS. 28A-D shows time courses of APs for baseline (control) (FIG. 28A), with Isop treatment (FIG. 28B), PLN KD (=Kmf 25%) (FIG. 28C), and combined Isop treatment and PLN KD (PLN KD+Isop) (FIG. 28D). FIGS. 28E-F shows the incidence of delayed afterdepolarization (DAD) and triggered Action Potential (tAP) (FIG. 28E) and early afterdepolarization (EAD) (FIG. 28F) detected in the human atrial myocyte (HAM) populations for Isop and various degrees of PLN KD (Kmf varied from 25% to 75%) conditions.

FIGS. 29A-G shows the effects of PLN KD (Kmf 25%) and Isop on the generation of triggered activity in heterogeneous human atrial tissue. FIG. 29A shows the spatial distribution of DADs and tAPs in the atrial tissue with reduced cell-to-cell coupling for PLN KD (Kmf 25%) and after Isop treatment. FIG. 29B shows the total number of DADs and tAPs detected in the atrial tissue after each perturbation with normal or reduced cell-to-cell coupling. FIGS. 29C-D shows superimposed traces of APs from two regions (marked in FIG. 29A) of the atrial tissue with reduced cell-to-cell coupling for each perturbation. FIG. 29E shows the spatial distribution of DADs and tAPs in the atrial tissue for PLN KD (Kmf 25%) and after Isop treatment. FIGS. 29F-G shows superimposed traces of APs from two regions (marked in FIG. 29E) of the atrial tissue with normal cell-to-cell coupling for each perturbation.

FIG. 30A shows a schematic describing the logistic regression analysis approach to identify the mechanisms underlying the generation of DADs in HAMs. FIG. 30B shows logistic regression analysis of DAD incidence in the context of moderate PLN knockdown (Kmf 50%) revealed influence of model parameters on the genesis of DADs in the population of HAMs in response to the pacing-pause protocol. Positive coefficients indicate that increasing the associated parameters promotes DAD production, and vice versa.

FIG. 31A shows histograms showing that the percentage of irregularly beating (AI>20) ACMs co-cultured with fibroblasts and treated with isoproterenol, is increased when transfected with siPLN and NCX as compared to siPLN alone. FIG. 31B shows representative AP peak trains for siControl, PLN siPLN+NCX conditions in ACMs co-cultured with fibroblasts and treated with isoproterenol. Arrowheads show examples of irregular beat to beat intervals in arrhythmically beating ACMs.

FIG. 32A shows mean Systolic Interval (SI) in response to cardiac KD of the plasma membrane Na+/Ca2+ exchanger NCX, SclA, and combined SclA+NCX KD. Co-KD caused a greater decrease in SI than did single KD alone (p<0.05, Wilcoxon ranked sum test). FIG. 32B shows representative m-modes showing effects of KD on SI.

FIG. 33A shows histograms showing that the percentage of irregularly beating (AI>20) ACMs co-cultured with fibroblasts and treated with isoproterenol, is decreased when treated with verapamil (30 nM) as compared to DMSO. FIG. 33B shows representative AP peak trains and AI values for siPLN; siPLN+verapamil (30 nM); siControl conditions in ACMs co-cultured with fibroblasts and treated with Isop. Arrowheads show examples of irregular beat-to-beat intervals in arrhythmically beating ACMs. ****P<0.0001 (KS-D).

FIGS. 34A-D shows schematics summarizing how the integrated multiplatform approach of the present disclosure enables the HT identification and characterization of AF-associated genes and mechanisms, using model systems with human, adult, whole organ and atrial relevance.

FIGS. 35A-C shows normalized histogram plots illustrating the distribution of APD90 for the human atrial population of isolated cardiomyocytes (FIG. 35A) and coupled cardiomyocytes in tissue with reduced (FIG. 35B) or normal (FIG. 35C) cell-to-cell coupling. The mean and SD values are indicated in each panel.

### DETAILED DESCRIPTION

Atrial fibrillation (AF) is a common and genetically inheritable form of cardiac arrhythmia; however, it is currently not known how these genetic predispositions contribute to the initiation and/or maintenance of AF-associated phenotypes. One major barrier to progress is the lack of experimental systems enabling to rapidly explore gene function on rhythm parameters in models with human atrial and whole organ relevance. The disclosure provided herein describes a multi-model platform enabling 1) the high-throughput characterization of gene function on action potential duration and rhythm parameters using human iPSC-derived atrial-like cardiomyocytes and the Drosophila heart model, and 2) the validation of the findings using computational models of human adult atrial myocytes and tissue. Mechanistically, the present disclosure reveals that Phospholamban regulates rhythm homeostasis by functionally interacting with L-type calcium channels and NCX. In summary, the novel multi-model system approach illustrated herein paves the way for the discovery and molecular delineation of gene regulatory networks controlling atrial rhythm with application to AF.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. When ranges are used herein for physical properties, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### In Vitro-Generated Cardiomyocyte Model of Cardiac Rhythm Disorders

In an aspect, the present disclosure describes an in vitro-generated cardiomyocyte. The in vitro-generated cardiomyocyte is generated from a reprogrammed cell in vitro. The in vitro-generated cardiomyocyte comprises at least one gene associated with a cardiac rhythm disorder having an altered expression status. The in vitro-generated cardiomyocyte displays a phenotype associated with the cardiac rhythm disorder. In some embodiments, the cardiac rhythm disorder is atrial fibrillation (AF). In some embodiments, the cardiomyocyte is an atrial-like cardiomyocyte (ACM).

In some embodiments, the gene associated with the cardiac rhythm disorder is selected from a group comprising GATA5, GATA6, PITX2, KCNA5, GATA4, KCNJ5, HCN4, GJA1, TBX5, SYNE2, NKX2-6, SH3PXD2A, KCNN3, NPPA, ZFHX3, NKX2-5, HAND2, GJA5, KCND3, and PLN. In some embodiments, the gene associated with the cardiac rhythm disorder is selected from a group comprising but not limited to ABCC9, C9ORF3, CAND2, CAV1, CEP68, CUX2, GATA1, GATA2, GATA3, GATA4, GATA5, GATA6, GJA1, GJA5, GREM2, Hand, HAND2, HCN2-4, JPH2, KCNA1-5, KCND1-3, KCNE1-5, KCNH2, KCNJ10, KCNJ12, KCNJ15, KCNJ18, KCNJ2, KCNJ4, KCNJ5, KCNJ8, KCNK3, KCNMA, KCNN1-3, KCNN2, KCNN3, KCNQ1, LMNA, MYH6, MYL4, NEBL, NEURL, NKX2.1, NKX2.4, NKX2.5, NKX2-5, NKX2-6, NPPA, PITX2, PLN, PRRX1, Ptx1-3, RYR2, SCN1-5, SH3KBP1, SH3PXD2A, SHOX2, SOX5, SYNE1, SYNE2, SYNPO2L, TBX2, TBX3, TBX5, TBX6, TWIST1, TWIST2, ZFH2-4, and ZFHX3.

In some embodiments, the phenotype associated with the cardiac rhythm disorder is an alteration in a cardiac rhythm parameter. The cardiac rhythm parameter may be selected from a group comprising action potential duration (APD), systolic interval, beat rate, beat refractory period, peak-to-peak interval, early afterdepolarization, delayed afterdepolarization, and Arrythmia Index (AI) value. In some embodiments, the phenotype is an AI value greater than 20. In some embodiments, the alteration in the cardiac rhythm parameter is a change in the APD75 value, wherein the APD75 value is APD measured at 75% repolarization. In some embodiments, the alteration in the cardiac rhythm parameter is a change in the APD90 value, wherein APD90 is APD measured at 90% repolarization. In some embodiments, the alteration in the cardiac rhythm parameter is a shortening of the APD as compared to a reference cardiomyocyte. In some embodiments, the alteration in the cardiac rhythm parameter is an increase in the beat refractory period as compared to a reference cardiomyocyte. In some embodiments, the alteration in the cardiac rhythm parameter is an increase in beat rate as compared to a reference cardiomyocyte. In some embodiments, the alteration in the cardiac rhythm parameter is a reduction in the systolic interval as compared to a reference cardiomyocyte. In some embodiments, the alteration in the cardiac rhythm parameter is a shortening of the Ca2+ transient duration as compared to a reference cardiomyocyte.

In some embodiments, the altered expression status is overexpression of the at least one gene associated with the cardiac rhythm disorder as compared to the expression level of the gene in a reference cardiomyocyte. Genes associated with a cardiac rhythm disorder may be selected from the group comprising but not limited to ABCC9, C9ORF3, CAND2, CAV1, CEP68, CUX2, GATA1, GATA2, GATA3, GATA4, GATA5, GATA6, GJA1, GJA5, GREM2, Hand, HAND2, HCN2-4, JPH2, KCNA1-5, KCND1-3, KCNE1-5, KCNH2, KCNJ10, KCNJ12, KCNJ15, KCNJ18, KCNJ2, KCNJ4, KCNJ5, KCNJ8, KCNK3, KCNMA, KCNN1-3, KCNN2, KCNN3, KCNQ1, LMNA, MYH6, MYL4, NEBL, NEURL, NKX2.1, NKX2.4, NKX2.5, NKX2-5, NKX2-6, NPPA, PITX2, PLN, PRRX1, Ptx1-3, RYR2, SCN1-5, SH3KBP1, SH3PXD2A, SHOX2, SOX5, SYNE1, SYNE2, SYNPO2L, TBX2, TBX3, TBX5, TBX6, TWIST1, TWIST2, ZFH2-4, and ZFHX3.

In some embodiments, the altered expression status is reduced expression of the at least one gene associated with the cardiac rhythm disorder as compared to the expression level in a reference cardiomyocyte. In some embodiments, the in vitro-generated cardiomyocyte further comprises a nucleic acid molecule capable of modulating the expression of the at least one gene associated with the cardiac rhythm disorder. The nucleic acid molecule may comprise but is not limited to small interfering ribonucleic acid (siRNA), short hairpin RNA (shRNA), a nucleic acid expressing CRISPR/Cas9, a nucleic acid expressing a TALEN, a nucleic acid expressing a zinc finger nuclease, a nucleic acid expressing a meganuclease, a nucleic acid expressing an endonuclease, or any combination thereof.

In some embodiments, the reprogrammed cell is a cardiac progenitor cell. In some embodiments, the cardiac progenitor cell overexpresses Id1. In some embodiments, the reprogrammed cell is an induced pluripotent stem cell (iPSC). In some embodiments, the reprogrammed cell is a stem cell. In some embodiments, the cardiomyocyte expresses one or more genes selected from a group comprising but not limited to NR2F2, TBX5, ZNF385B, KCNJ3, KCNA5, NPPA, NPPB, EGR 1/2 and PDGFRA.

In an aspect, the present disclosure describes a cell population comprising at least two in vitro-generated cardiomyocytes of the present disclosure. In some embodiments, the percentage of the cell population that exhibits an AI value of at least 20 is greater than 5%, 10%, 15%, 20%, 25%, 30%, 35%, 40%, 45%, 50%, 55%, 60%, 65%, 70%, 75%, 80%, 85%, 90%, 95%, 99%, or any integer in between. In some embodiments, the APD75 value measured using a Kolmogorov-Smirnov scale is at least 0.1, 0.2, 0.3, 0.4, 0.5, 0.6, 0.7, 0.8, or 0.9 when compared to the APD75 value of a reference cardiomyocyte cell population.

### In Vitro Cardiac Disorder Screening

In an aspect, the present disclosure describes a cell co-culture model of cardiac fibrosis comprising the in vitro-generated cardiomyocyte of the present disclosure and a fibroblast cell. In some embodiments, the ratio of the fibroblast cell to the cardiomyocyte cell is 3:1. In some embodiments, the ratio of the fibroblast cell to the cardiomyocyte is 2:1, 1:1, or 0.5:1.

In an aspect, the present disclosure describes a cell co-culture model of cardiac arrhythmia comprising the in vitro-generated cardiomyocyte of the present disclosure and a pharmaceutical compound. In some embodiments, the pharmaceutical compound is isoproterenol. In some embodiments, the pharmaceutical compound is dofetilide.

In an aspect, the present disclosure provides a method for screening a candidate agent for the treatment of a cardiac rhythm disorder comprising contacting the cardiomyocyte of the present disclosure with the candidate agent and detecting an effect of the candidate agent on the phenotype associated with the cardiac rhythm disorder. In some embodiments, the method further comprises culturing the cardiomyocyte with a fibroblast cell prior to contacting with the candidate agent. In some embodiments, the method further comprises labeling the cardiomyocyte with a voltage dye or a nuclear dye. In some embodiments, the method further comprises quantifying the voltage-dependent fluorescence variation over time. In some embodiments, the method further comprises automatically processing the action potential trace. In some embodiments, the method further comprises measuring parameters selected from the group comprising: APD-10, 25, 50, 75, 90; T25-75, T75-25; Vmax up and down; beat rate; peak-to-peak interval; and rhythm regularity index. In some embodiments, the candidate agent is a nucleic acid. In some embodiments, the candidate agent is a small molecule. In some embodiments, the candidate agent is a protein. In some embodiments, the nucleic acid recognizes the gene phospholamban (PLN). In some embodiments, the nucleic acid recognizes the gene NCX. In some embodiments, the candidate agent is isoproterenol. In some embodiments, the candidate agent is dofetilide. In some embodiments, the candidate agent is verapamil. In some embodiments, the detecting is conducted at single cell resolution.

### Cardiac Disorder Screening in Human Subjects

In an aspect, the present disclosure provides a method of determining an increased risk for atrial fibrillation (AF) in a human subject comprising collecting a biological sample from the human subject and determining by an assay a level of a gene or gene product associated with AF in the biological sample. In some embodiments, the assay further comprises contacting the biological sample with a reagent that recognizes the gene or gene product associated with AF. In some embodiments, the biological sample is blood from the human subject. In some embodiments, the biological sample is a DNA sample. In some embodiments, the assay comprises genome sequencing of the human subject. In some embodiments, the assay comprises a proteomic assay. In some embodiments, the method further comprises administering a therapeutic agent to the human subject, wherein the therapeutic agent is configured to mitigate or alleviate one or more symptoms of AF in the human subject. In some embodiments, the gene is phospholamban (PLN). In some embodiments, the gene is NCX. In some embodiments, the gene product is a L-type Calcium channel. In some embodiments, the therapeutic agent is verapamil.

### High Throughput Multiplatform Cardiac Disorder Screening

In an aspect, the present disclosure provides a method for high-throughput identification of a gene underlying a cardiac rhythm disorder comprising evaluating the effect of the loss-of-function and gain-of-function of the gene on the in vitro-generated cardiomyocyte of the present disclosure, evaluating the effect of the loss-of-function and gain-of-function of the gene on a Drosophila heart, or computational modeling of the effect of knockdown of the gene on a computational model of heterogenous adult human atrial myocytes (HAMs). In some embodiments, the cardiac rhythm disorder is AF. In some embodiments, the evaluating comprises measuring a phenotype associated with the cardiac rhythm disorder, wherein the phenotype is an alteration in a cardiac rhythm parameter. In some embodiments, the cardiac rhythm parameter is selected from a group comprising action potential duration (APD), systolic interval, beat rate, beat refractory period, peak-to-peak interval, early afterdepolarization, delayed afterdepolarization, and Arrythmia Index (AI) value.

In some embodiments, the evaluating comprises measuring parameters of Drosophila heart function selected from the group comprising: heart period (R-R interval), systolic interval (SI), arrhythmicity, fractional shortening, and contractility. In some embodiments, the modeling further comprises simulating parameters selected from a group comprising: affinity of the sarco/endoplasmic reticulum Ca2+-ATPase (SERCA) for cytosolic Ca2+, maximum ion channel conductances, rates for membrane transporters, and Ca+2 handling fluxes. In some embodiments, the method further comprises simulating the parameters on a cell population basis. The cell population may comprise at least 100 HAMs, at least 200 HAMs, at least 300 HAMs, at least 400 HAMs, 500 HAMs, at least 600 HAMs, at least 700 HAMs, at least 800 HAMs, at least 900 HAMs, or at least 1000 HAMs.

The following examples further illustrate aspects of the disclosure, but should not be construed as in any way limiting its scope. All references cited herein are each hereby incorporated by reference in their entirety, For example, Kervadec A, et al. Multiplatform modeling of atrial fibrillation identifies phospholamban as a central regulator of cardiac rhythm. Dis Model Mech. 2023 Jul. 1; 16 (7): dmm049962. doi: 10.1242/dmm.049962. Epub 2023 Jul. 17. PMID: 37293707; PMCID: PMC10387351 is incorporated herein by reference in its entirety.

### EXAMPLES

### Example 1: Integrated Multi-Modal Systems Platform Identifies Rhythm-Regulating Genes

In this example, a novel phenotypic platform was established to phenotypically assess atrial fibrillation (AF) associated genes, enabling the study of gene function on action potential duration (APD) and rhythm parameters in high throughput (HT) in atrial-like cardiomyocytes (ACMs).

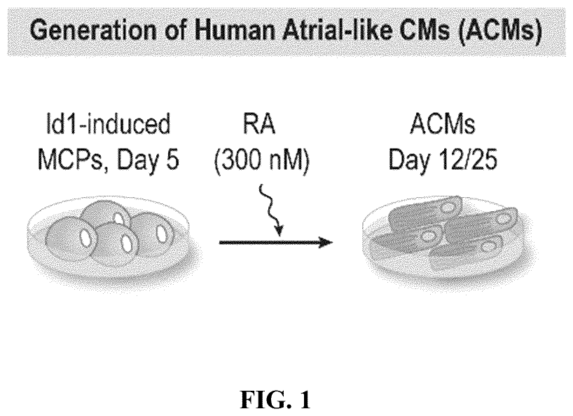

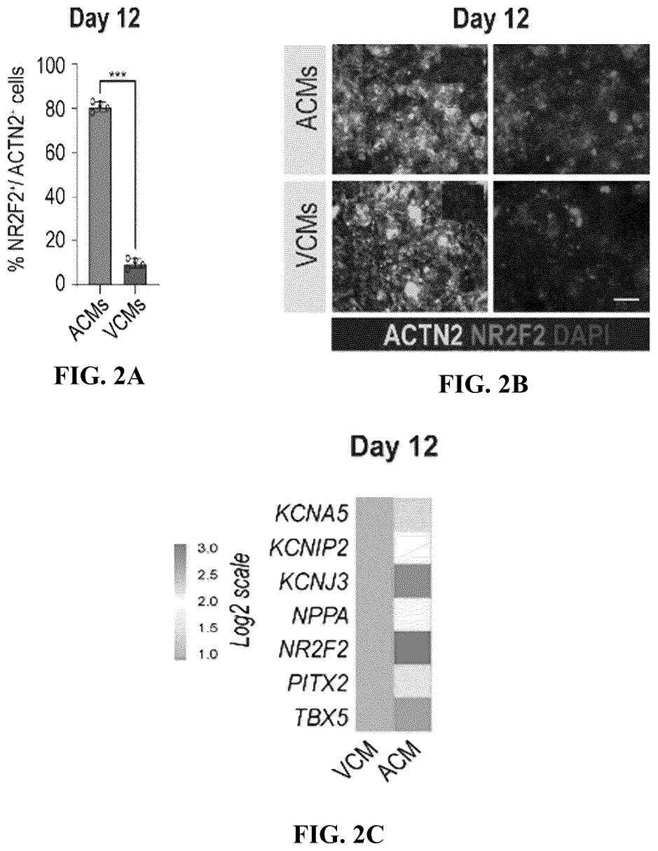

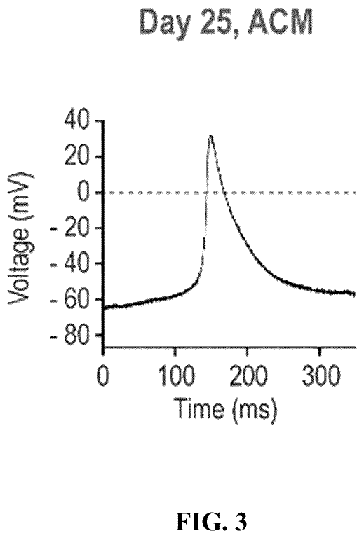

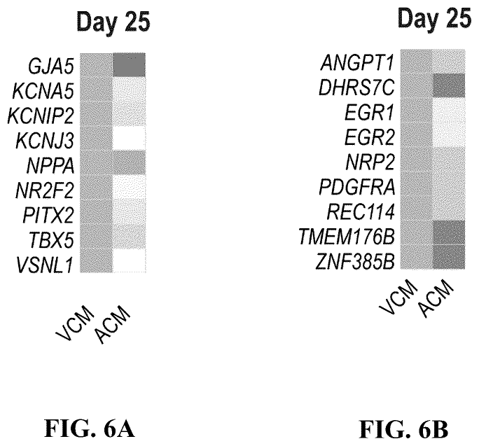

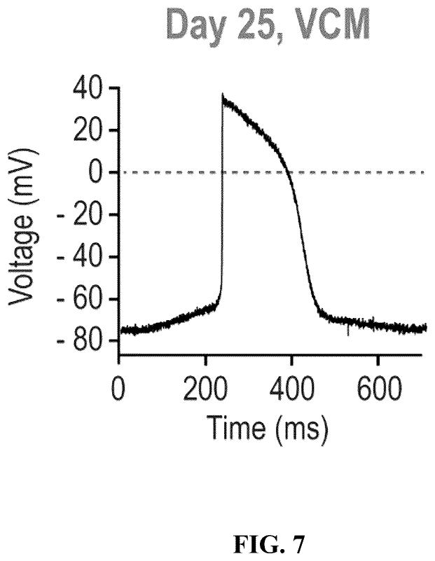

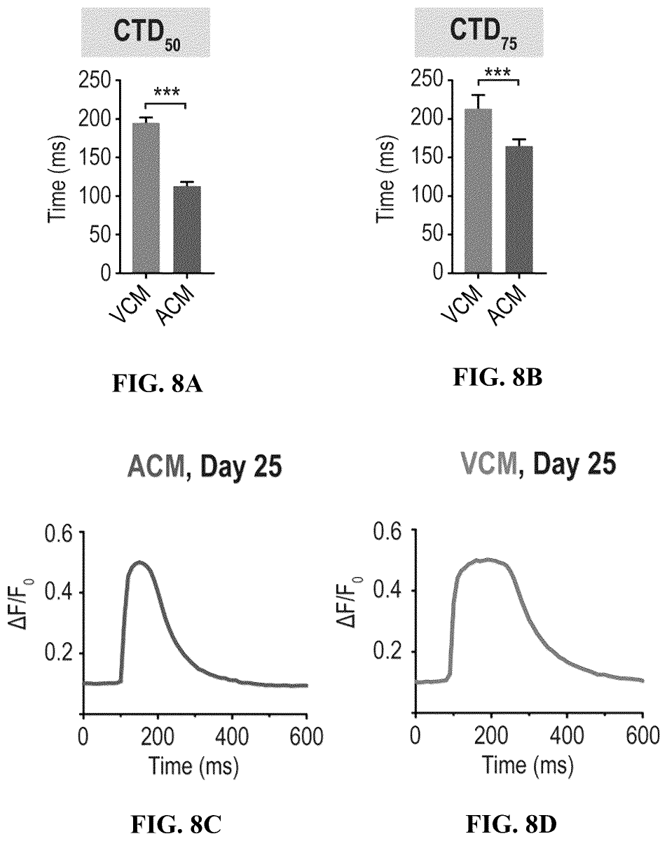

To study the molecular basis of chamber-specific electrical disorders such as atrial fibrillation, Id1-overexpressing cardiac progenitors (CPs) were used to generate ACMs (Cunningham et al., 2017, and Yu et al., 2018). Treatment of Id1-induced CPs with a single dose of retinoic acid (300 nM) efficiently promoted the generation of atrial-like, NR2F2+ beating CMs (˜80% were NR2F2+, ACTN2+) (FIG. 1 and FIGS. 2A-B). Consistent with an atrial-like identity, induced ACMs also expressed atria-enriched (Devalla et al., 2015; Uhlen et al., 2015) transcription factors (NR2F2, TBX5, ZNF385B), ion channel genes KCNA5 (encoding Kv1.5) and KCNJ3 (encoding Kir 3.1), ligands (NPPA, NPPB) and receptors (EGR1/2, PDGFRA), at both day 12 and at day 25 of differentiation (FIG. 2C and FIGS. 6A-B). Electrophysiologically, ACMs typically generated short (˜120 ms) and triangular action potentials (FIG. 3) while untreated Id1-CPs generated CMs displayed longer action potential (˜200 ms) with a plateaued phase 2 (FIG. 7), reminiscent of a ventricular-like identity (Ng et al., 2010). Moreover, and consistent with an atrial cell fate, ACMs also displayed shorter calcium transient durations (CTD₅₀ and CTD₇₅) as compared to ventricular CMs (VCMs) (FIGS. 8A-D) (Ng et al 2010).

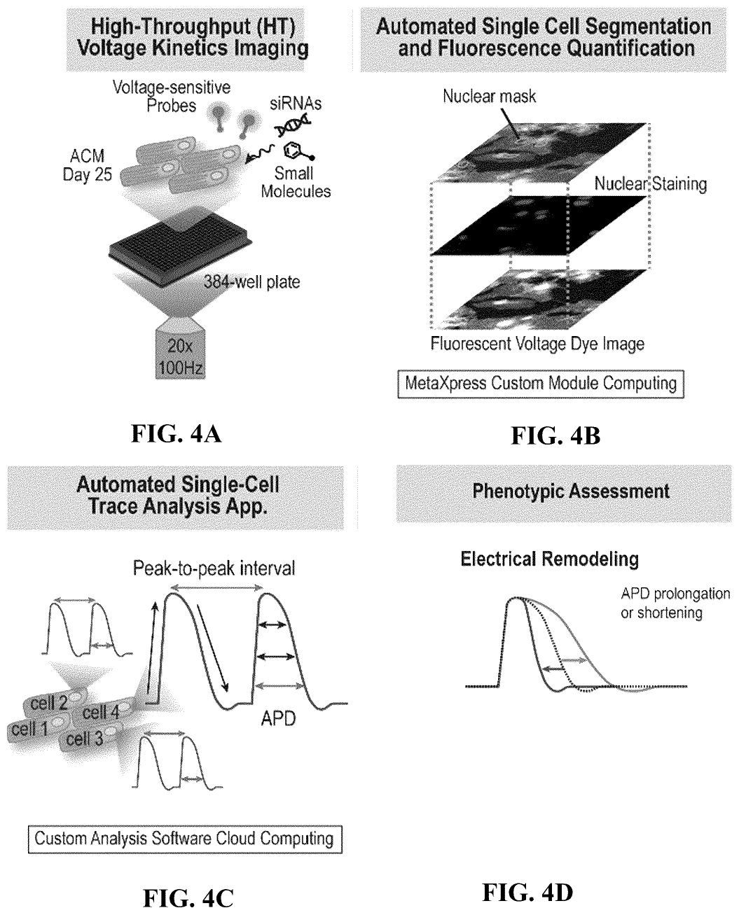

Next, to facilitate the characterization of AF-associated arrhythmia phenotypes in ACMs, an imaging platform was developed that automatically tracks and quantifies action potential (AP) and rhythm parameters in HT with single cell resolution (FIGS. 4A-D). To retrieve AP and rhythm parameters, ACMs of the present disclosure were co-labeled with a voltage dye (VF2.1Cl) and a nuclear dye (Hoechst 33258) (McKeithan et al., 2017). For each condition, one image of the Hoechst dye was collected followed by a 5-second acquisition of the voltage dye channel at 100 Hz. Next, using a custom algorithm developed on the ImageXpress®, each cell in the field of view was segmented using the Hoechst topological information and each cell mask was propagated to the “voltage dye channel”, thereby enabling the quantification of voltage-dependent fluorescence variation over time with single-cell resolution. To retrieve the electrophysiological parameters, a cloud-based trace analysis application was developed that automatically processes each AP trace and retrieves median and standard deviation values, for APD-10, 25, 50, 75, 90; T25-75, T75-25; Vmax up and down; beat rate; peak-to-peak interval; and rhythm regularity index; for each cell. This platform enabled the automatic recording, quantification, and analysis of AP and rhythm parameters in less than 2 minutes per condition.

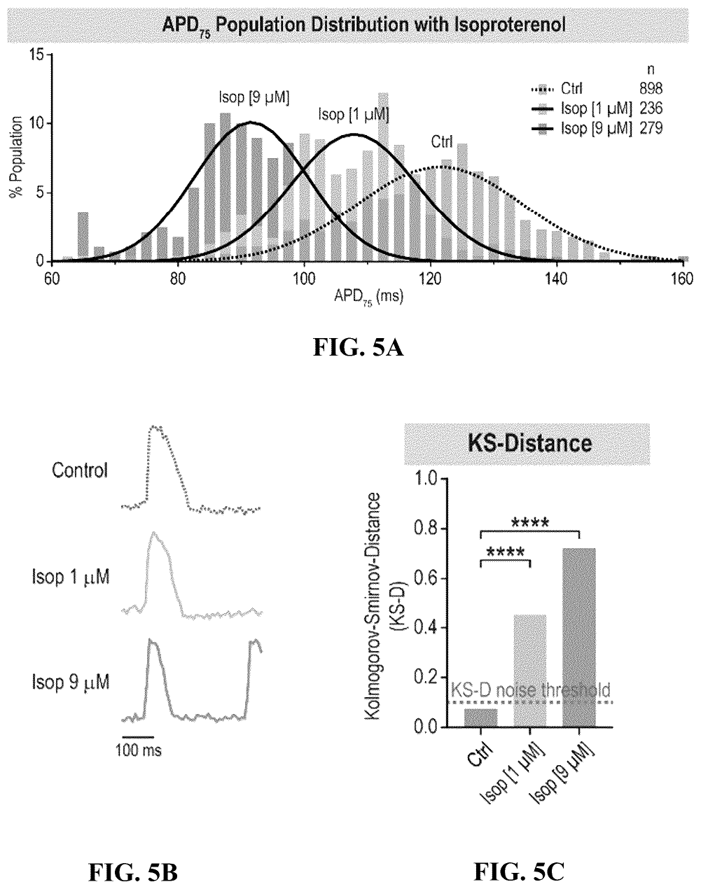

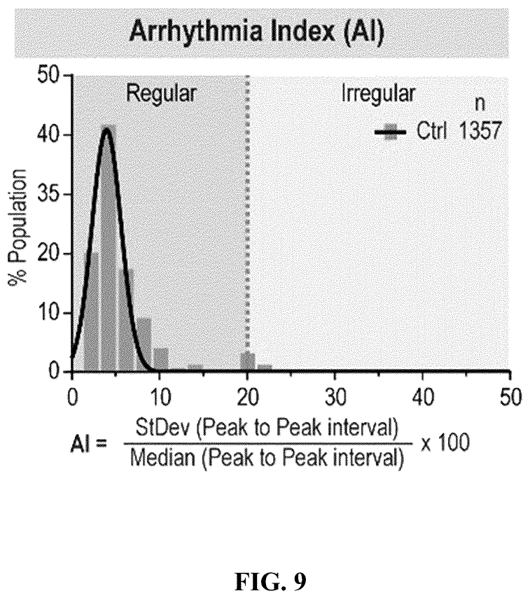

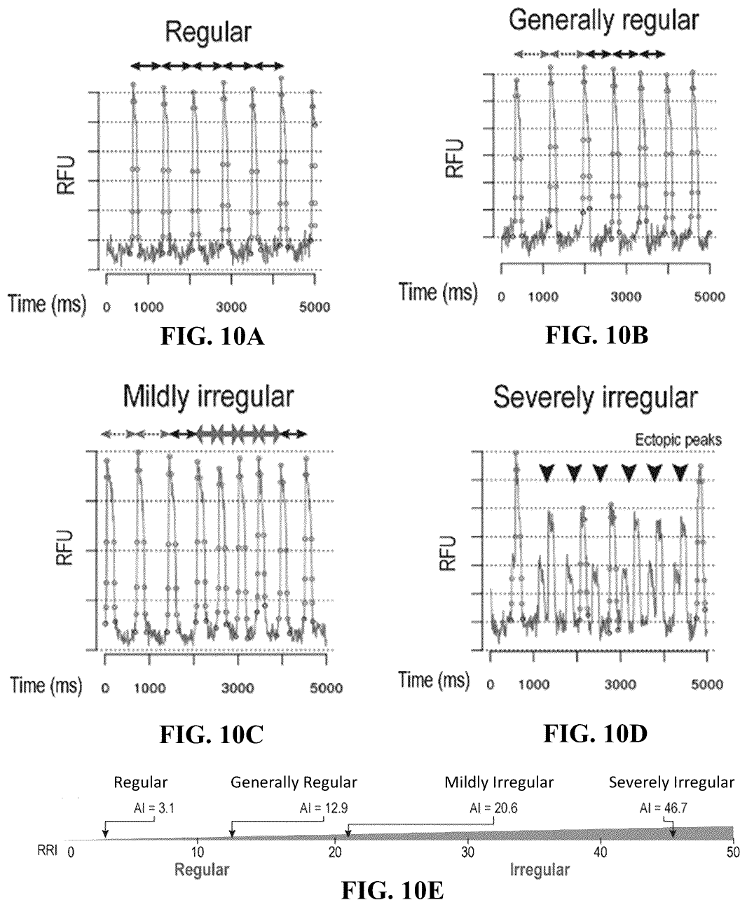

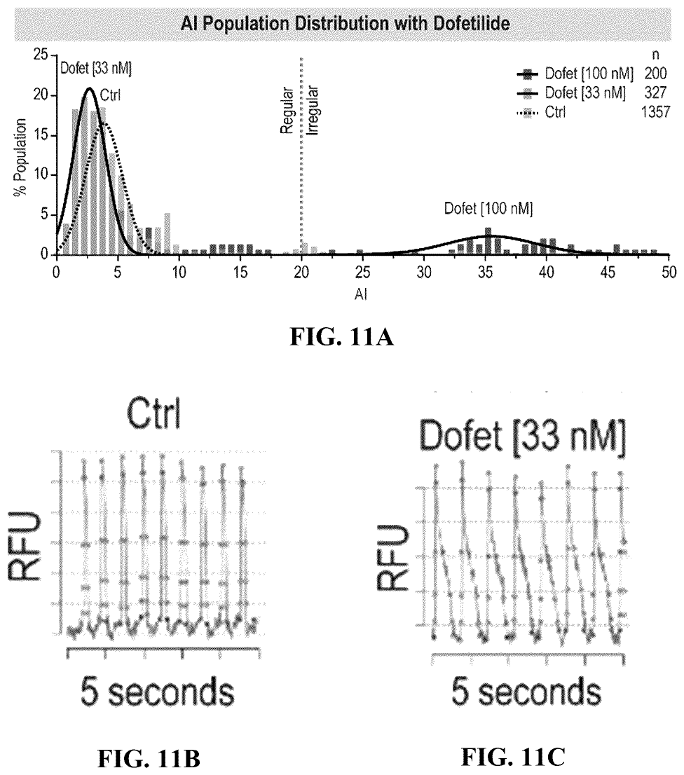

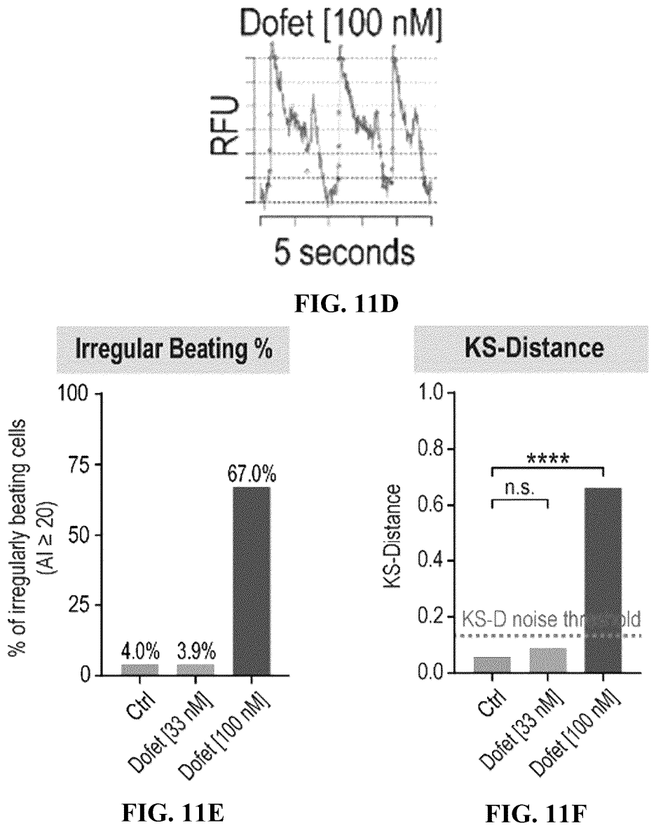

To test the platform's ability to identify APD modulators, ACMs of the present disclosure were infused with isoproterenol, a non-selective β-adrenergic agonist, known to both shorten APD and increase beat rate in hiPSC-CMs. Escalating doses of isoproterenol caused a dose-dependent shortening of median APD75 values, from 121.3 ms (untreated) to 108.6 ms (1 μM) and 91.2 ms (9 μM) (FIGS. 5A-B). To measure whether isoproterenol treatment had a significant effect on APD at the whole cell population level, the Kolmogorov-Smirnov test was used (KS-D) (Dal Molin et al., 2017; Delmans and Hemberg, 2016; Feng et al., 2009; and Gaber et al., 2013) which enables to quantify and compare the distributional differences of binary features such as APD₇₅ or beat rate. Consistent with median APD₇₅ values, escalating doses of isoproterenol (1 and 9 μM) led to an increase of APD₇₅ KS-D value to ˜0.4 and 0.7 respectively, as compared to control (FIG. 5C). Similarly, to quantify cellular manifestations of arrhythmia, an arrhythmia index (AI) was developed that quantifies beat-to-beat interval irregularities as a metric for arrhythmically beating cells (FIG. 9). In this context, AP trains with AI values lower than 20 were determined to describe regular beating patterns, while AP train with AI values >20 mark were determined to describe irregular beating patterns (FIGS. 10A-E). Finally, to benchmark the platform for arrhythmia-associated phenotypes, the role of dofetilide, a class III anti-arrhythmic that selectively blocks the rapid component of the delayed rectifier outward potassium, was tested. At therapeutic doses, dofetilide prolongs APD and subsequently increases the refractory period, thereby mediating its anti-arrhythmic effect (Geng et al., 2020). Conversely, at higher doses, dofetilide increases the incidence of arrhythmia phenotypes such as early afterdepolarizations (EADs) (Jaiswal and Goldbarg, 2014; McKeithan et al., 2017). Consistent with these observations, ACMs treated with a low dose of dofetilide (33 nM) displayed a reduced AI as compared to control, while high dose of dofetilide (100 nM) dramatically increased the percentage of cells with AI values >20 (from 4% to 67%) and associated KS-D value of 0.7 (FIGS. 11A-F). Collectively, these data show that this new phenotypic platform enables the HT and automated quantification of APD and rhythm parameters in ACMs with single cell resolution.

FIGS. 1-5 show an overview of the ACM platform of the present disclosure. FIG. 1 shows a schematic representation of an ACM differentiation protocol in which day 5 cardiac progenitors were treated with 300 nM retinoic acid to promote atrial differentiation and subsequently cultured until either day 12 or 25. FIG. 2A shows a histogram showing ˜80% of ACMs are NR2F2+/ACTN2+ at day 12 following retinoic acid treatment. FIG. 2B shows immunofluorescence images showing expression of NR2F2+ and ACTN2+ in ACMs and VCMs stained with DAPI. FIG. 2C shows a heatmap of atrial genes enriched in ACMs as compared to VCMs. FIG. 3 shows a graph of an atrial-like triangular-shaped action potential collected via patch-clamp experiments in ACMs at day 25. FIGS. 4A-D shows a schematic representation of a single cell and high throughput platform to measure APD parameters in ACMs. FIG. 5A shows a population distribution of APD₇₅ values from ACMs treated with escalating doses of isoproterenol, which shows a dose dependent APD shortening. FIG. 5B shows single AP traces of Median APD₇₅ in a control condition, 1 μM isoproterenol condition, and a 9 M isoproterenol condition. FIG. 5C shows a histogram showing Kolmogorov-Smirnov Distance (KS-D) values for control, 1 μM isoproterenol, and 9 μM isoproterenol treated ACMs.

FIGS. 6-8 show gene expression and functional characterization of ACMs. FIGS. 6A-B shows heatmaps of atrial genes enriched in day 25 ACMs as compared to VCMs. FIG. 7 shows an action potential collected via patch-clamp experiments in VCMs, which generate action potentials with ventricular characteristics, including a long phase 2. FIGS. 8A-B shows histograms quantifying average CTD50 and CTD75, respectively, demonstrating that ACMs display shorter calcium transients than VCMs (P-value ***<0.001). FIGS. 8C-D shows representative calcium transient traces for ACMs and VCMs, respectively.

FIGS. 9-11 show quantification of rhythm parameters in ACMs. FIG. 9 shows a histogram representing a population distribution of Arrhythmia Index (AI) values for control cells (n=1357) in which AI values <20 correspond to a not arrhythmic phenotype while AI values >20 correspond to an arrhythmic phenotype. The equation used to calculate AI values in cells is shown below the graph. FIGS. 10A-E shows examples of peak trains from ACMs with regular (FIG. 10A), generally regular (FIG. 10B), mildly irregular (FIG. 10C), and severely irregular (FIG. 10D) with their respective AI scores shown in FIG. 10E, wherein values less than 20 correspond to a regular phenotype and values greater than 20 correspond to an irregular phenotype. FIG. 11A shows a population distribution histogram representing the distribution of AI values for ACMs treated with increasing doses of Dofetilide. FIGS. 11B-D shows representative peak trains of control ACMs (FIG. 11B), ACMs treated with 33 nM Dofetilide (FIG. 11C), and ACMs treated with 100 nM Dofetilide (FIG. 11D). FIG. 11E shows a histogram representing the proportion of arrhythmic cells (AI>20) in control ACMs (4.0%), and ACMs treated with 33 nM (3.9%) and 100 nM (67.0%) Dofetilide. FIG. 11F shows a histogram representing quantification of KS-D between control or Dofetilide-treated ACMs conditions. P-value ***<0.0001.

### Example 2: APD and Rhythm Parameters were Measured in HT in a Drosophila Cardiac Platform

In this example, the HT phenotypic platform of the present disclosure was used to assess APD and rhythm parameters in flies.

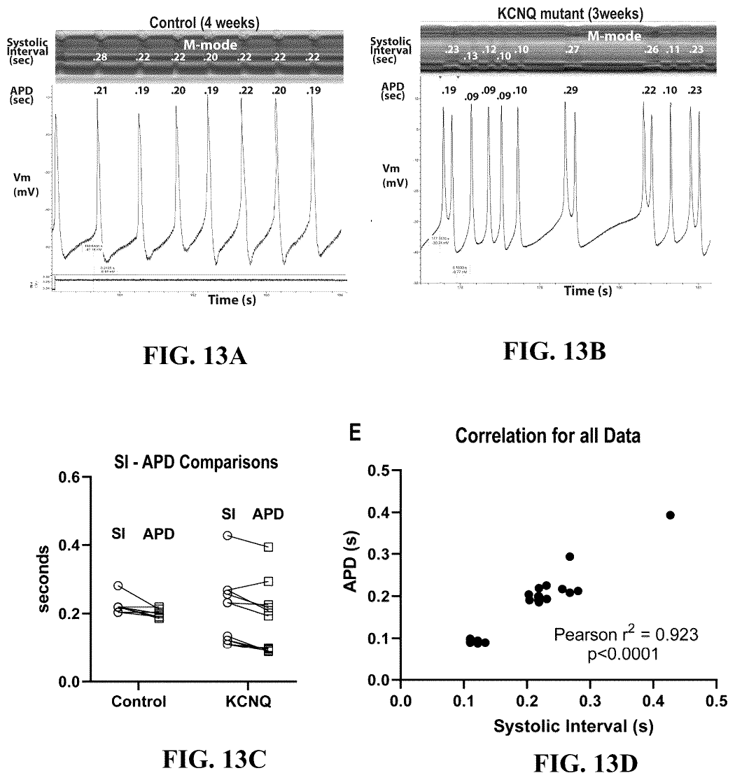

To assess AF-associated mechanisms at the whole organ level using the Drosophila model, high-speed video recording of heart movements was used in in situ preparations. Heart function was quantified (Fink et al., 2009; Vogler and Ocorr, 2009) providing precise measurements of heart period (R-R interval), systolic interval (SI), as well as arrhythmicity and fractional shortening/contractility in a functioning heart. Most of the key cardiac ion channels present in human hearts are also present and functional in the fly heart (Ocorr et al., 2007c; Ocorr et al., 2017) (Table 1). Importantly, simultaneous optical and intracellular recordings demonstrated a direct 1:1 correlation between myocardial cell depolarization and heart wall movement. It is important to note that the fly heart is composed of a single layer of myocardial cells and any heart wall movement is an immediate reflection of the contractile state of component myocardial cells. Thus, APD was quantified and the corresponding systolic interval (SI) from simultaneous electrical and optical recordings from hearts of middle-aged wildtype controls and KCNQ mutants. A strong correlation (r=0.96, p<0.0001) was found between APD and SI (FIGS. 13A-D). Therefore, cardiac contraction and relaxation movements were used as surrogates for APD (Cammarato et al., 2015). In this context, cardiac-specific gene KD was achieved using a Gal-4 based system (Brand and Perrimon, 1993) that drives expression of double-stranded RNA interference (dsRNAi) in a cardiac-specific manner. Since AF is an aging-related disease, the fly provides an opportunity to examine effects of cardiac gene KD at young, middle, and old ages (˜1, 3, and 5 weeks, respectively). Finally, ˜75% of human disease-causing genes are represented in the fly, usually as single copies, and often cause remarkably similar disease phenotypes (Bier, 2005; Bier and Bodmer, 2004). For example, flies with mutations in the KNCQ gene exhibited a torsades de pointes-like phenotype, and age-dependent increases in arrhythmia in wildtype flies was associated with reduced expression of genes encoding KATP and KCNQ channels (Ocorr et al., 2007a). It was also found that electrical remodeling increased with age and was exacerbated by KCNQ and hERG mutations (Ocorr et al., 2017).

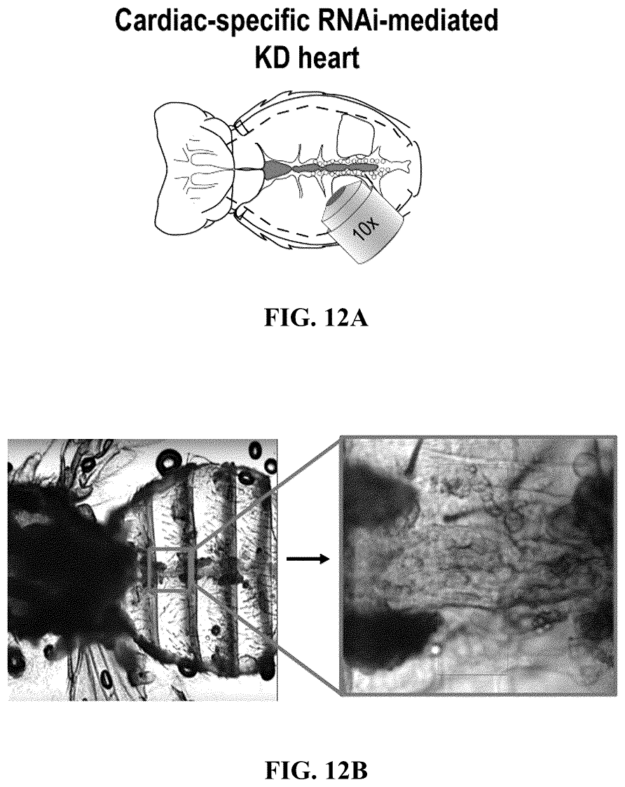

FIGS. 12-13 show the use of a fly heart platform for modeling AF-associated genes. FIG. 12A shows a schematic of the fly thorax and abdomen (heart tube is shown in center gray highlighted area above 10× objective). FIG. 12B shows an image of a semi-intact preparation (left) with a single cardiac chamber (gray box) shown at higher magnification to the right. FIGS. 13A-B shows simultaneous optical and electrophysiological recordings from beating hearts. M-modes from optical recordings are shown on the top with the corresponding Action Potential (AP) traces below. AP duration (APD) and Systolic Intervals are shown in seconds. The lower window in FIG. 13A shows the voltage trace generated by the image capture software that was used to synchronize the optical and electrical recordings. FIG. 13C shows systolic intervals paired with their corresponding APs. FIG. 13D shows the Pearson Correlation Coefficient for the combined data in FIG. 13C, which showed a significant correlation between SIs and APDs (r=0.96, p<0.0001).

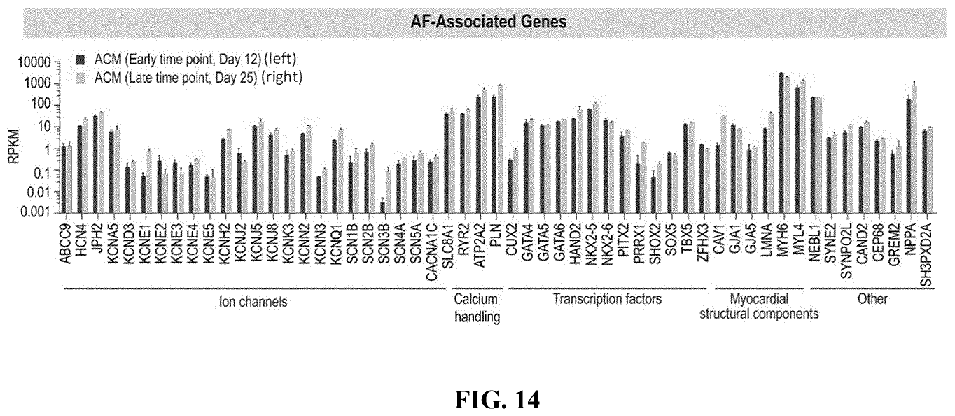

Table 1 below lists Human AF candidate genes tested in ACMs (FIG. 14) are shown with their Drosophila orthologs. Studies demonstrating the presence and/or function of these orthologs in the fly heart are listed and genes common to both ACMs and fly hearts are bolded. Data from tissue-specific RNA Seq analysis and curated in FlyAtlas2 (flyatlas.gla.ac.uk/FlyAtlas2) shows cardiac expression for approximately half of the genes in the table (indicated by +). Note that many of the ion channels and transcription factors (TF) that have been shown to be functional in the heart were not present in the Fly Atlas dataset and none were present in the cardiac proteomic analysis by Cammarato et al (2011, PMID: 21541028), likely because expression in the heart is too low relative to other structural genes (e.g. Myosin heavy chain, Mhc).

TABLE 1

| | | FLY | | |
|---|---|---|---|---|
| CATEGORY | GENE | ORTHOLOG | Reference | FlyAtlas2 |
| CHANNEL | ABCC9 | dSur | Akasaka, et al 2006- | − |
| | | | PMID: 16882722; | |
| | | | Eleftherianos et al, 2011- | |
| | | | PMID: 21719711 | |
| CHANNEL | HCN4 | Ih | Monier, et al, 2005- | + |
| | | | PMID 16284119 | |
| CHANNEL | JPH2 | junctophilin | | + |
| CHANNEL | KCNA5 | Shaker | Ocorr et al, 2017-PMID: | − |
| | | | 28542428 | |
| CHANNEL | KCND3 | Shal | Ocorr et al, 2017-PMID: | − |
| | | | 28542428 | |
| CHANNEL | KCNE1-5 | No ortholog | KCNQ works without | |
| | | | MinK | |
| CHANNEL | KCNH2 | seizure | Ocorr et al, 2017- | − |
| | | | PMID: 28542428 | |
| CHANNEL | KCNJ2, 5, | Irk | Ocorr et al, 2017- | − |
| | 8 | | PMID: 28542428 | |
| CHANNEL | KCNK3 | ork/sandman | LaLevee et al-PMID: | −/− |
| | | | 16890525 | |
| | | | Klassen et al-PMID: | |
| | | | 28328397 | |
| CHANNEL | KCNN2, 3 | SK | | + |
| CHANNEL | KCNMA | BK-not tested | Pineda et al,-PMID: | + |
| | | in ACMs? | 33629867 | |
| CHANNEL | KCNQ1 | KCNQ | Ocorr et al, 2007-PMID: | − |
| | | | 17360457 | |
| CHANNEL | RYR2 | RyR | Lin et al, PMID: | + |
| | | | 21493892 | |
| CHANNEL | SCN1-5 | nap | Dowse et al, PMID: | + |
| | | | 8719771 | |
| | | | Ganetsky, PMID: | |
| | | | 2420953 | |
| TF | CUX2 | cut | Blochlinger et al, PMID | − |
| | | | 8330519 | |
| | | | Zappia et al, PMID | |
| | | | 32815271 | |
| TF | GATA4/5/ | pnr/grn/ | Klinedinst & Bodmer, | +/+/+ |
| | 6 | GATAd | 2003-PMID: 12756184 | |
| TF | HAND2 | Hand | Kolsch and Paululat, 2002- | + |
| | | | PMID: 12424518 | |
| | | | Jonhson et al 2011- | |
| | | | PMID: 21965617 | |
| TF | NKX2-5/2- | tin | Bodmer et al 1990- | − |
| | 6 | | PMID: 7915669 | |
| TF | PITX2 | Ptx 1 | | − |
| TF | PRRX1 | CG9876 | | − |
| TF | SHOX2 | CG34367 | | − |
| TF | SOX5 | Sox102F | | + |
| TF | TBX5 | Bifid/ | Bi-Ahmad et al 2012- | −/−/−/− |
| | | Doc1/Doc2/ | PMID: 22814603 | |
| | | Doc3 | DOC-Reim et al 2003- | |
| | | | PMID: | |
| | | | 12783790 Berkeley | |
| | | | Drosophila Genome | |
| | | | Project | |
| TF | ZFHX3 | zfh2 | | − |
| MYOCARDIAL | CAV1 | No ortholog | | |
| MYOCARDIAL | GJA1 | CG11459/26- | Cammarato et al 2011- | + |
| | | 29-p/CG4847 | PMID: 21541028 | |
| MYOCARDIAL | GJA5 | No ortholog | | |
| MYOCARDIAL | LMNA | LamC/Lam | Cammarato et al 2011- | + |
| | | | PMID: 21541028 | |
| MYOCARDIAL | MYH6 | Mhc | Lovato et al, 2002- | + |
| | | | PMID: 12397110 | |
| | | | Cammarato et al, 2011- | |
| | | | PMID: 21541028 | |
| MYOCARDIAL | MYL4 | Mlc-c/Mlc1 | Cammarato et al 2011- | + |
| | | | PMID: 21541028 | |
| MYOCARDIAL | NEBL | Lasp | Cammarato et al 2011- | + |
| | | | PMID: 21541028 | |
| MYOCARDIAL | SYNE2 | Msp300 | Cammarato et al 2011- | + |
| | | | PMID: 21541028 | |
| MYOCARDIAL | SYNPO2L | CG1674 | Cammarato et al 2011- | + |
| | | | PMID: 21541028 | |
| OTHER | C9ORF3 | CG10602 | | + |
| OTHER | CAND2 | Cand1 | | + |
| OTHER | CEP68 | No ortholog | | |
| OTHER | GREM2 | No ortholog | | |
| OTHER | NEURL | neur | | + |
| OTHER | NPPA | No ortholog | | |
| OTHER | SH3PXD2A | cindr/Nipped- | Cammarato et al 2011- | +/+ |
| | | A | PMID: 21541028 | |

Table 2 below shows human AF candidate genes tested in the fly heart listed with their human orthologs and stock center ID number.

TABLE 2

| | Human | | | |
|---|---|---|---|---|
| Fly RNAi | Ortholog | Stock # | Stock #2 | Source |
| Bifid | TBX 2, 3 | 100598 | 330228 | VDRC |
| Cindr | SH3KBP1 | 38854 | 330411 | VDRC |
| Doc1 | TBX6 | 16746 | 104927 | VDRC |
| Doc2 | TBX6 | 103431 | | VDRC |
| Doc3 | TBX6 | 30550 | 104922 | VDRC |
| GATAd | GATA1 | 100389 | | VDRC |
| GATAe | GATA4 | 10418 | | VDRC |
| Grain | GATA2, 3 | 105192 | 330376 | VDRC |
| Hand | Hand | 23306 | 330058 | VDRC |
| Ih | HCN2-4 | 110274 | 29574 | VDRC |
| Irk1 | KCNJ2, 4, 12, 18 | 107389 | 28431 | VDRC |
| Irk2 | KCNJ2, 4, 12, 18 | 4341 | 108140 | VDRC |
| Irk3 | KCNJ10, 15 | 3886 | 101174 | VDRC |
| MSP300 | SYNE1 | 107183 | 25906 | VDRC |
| Pnr | GATA4, 5, 6 | 6224 | 101522 | VDRC |
| Pnr | GATA4, 5, 6 | 34659 | 33744 | BDSC |
| Ptx1 | Ptx1-3 | 19831 | 107785 | VDRC |
| SclA | PLN | 28957 | | BDSC |
| SclA/B | PLN | 62935 | 28957 | BDSC |
| Scro | NKX2.1, 2.4 | 33902 | 330398 | VDRC |
| Sh | KCNA1-5 | 23673 | 104474 | VDRC |
| Shal | KCND1-3 | 103363 | 330383 | VDRC |
| Sk | KCNN1-3 | 2855 | 7052 | VDRC |
| Sk | KCNN1-3 | 27238 | 53881 | BDSC |
| Tin | NKX2.5 | 190512 | 101825 | VDRC |
| Twist | TWIST1, 2 | 37091 | 37092 | VDRC |
| Zfh-2 | ZFH2-4 | 13305 | 110784 | VDRC |

### Example 3: Functional Screen of AF-Associated Genes Identifies PLN Loss of Function as Major Driver of APD and Contraction Intervals Shortening

In this example, gene expression in ACMs was assessed by RNA-sequencing (RNA-seq) to identify and target AF-associated genes.

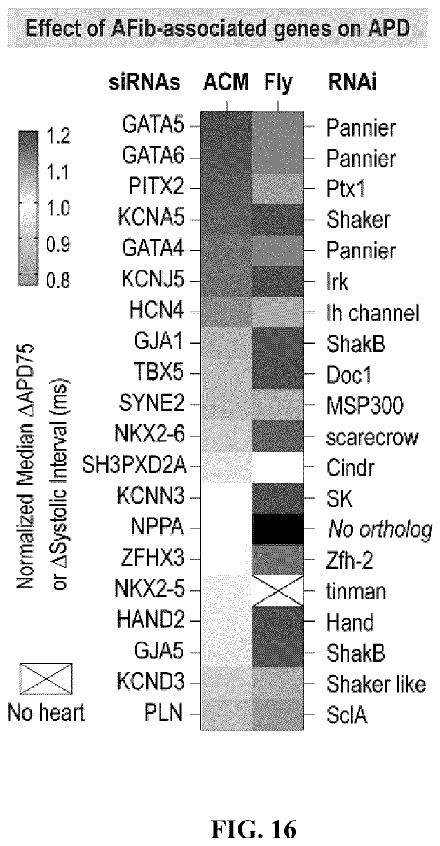

To evaluate the ability of the platform to identify AF-associated genes and mechanisms, the expression of genes previously associated with AF (Fatkin et al., 2017) was assessed by RNA-seq of day 12 and day 25 ACMs. The result revealed that most AF candidate genes were expressed in ACMs at moderate-to-high levels (from 0.1 to >100 RPKM) (FIG. 14) and most are also expressed in the fly heart (Table 1). Next, 20 genes were selected that had been identified in rare variant familial AF studies and/or as having SNPs reported in GWAS studies (TABLE 2 and Fatkin et al., 2017). To evaluate their effect on APD, siRNAs directed against these 20 AF-associated genes were transfected into day 25 ACMs and voltage variation was measured over time with single cell resolution. Remarkably, APD₇₅ population measurements revealed that 9 out of 20 siRNAs induced electrical remodeling (KS-D value >0.25, p-value <0.001) (FIG. 16, Table 3, Table 4, Table 5). Interestingly, the screen identified two phenotypes: prolongation or shortening of APD. Among these, down-regulation of GATA5, GATA6, PITX2, and KCNA5 significantly prolonged APD, whereas KD of PLN and KCND3 shortened APD.

In parallel, 24 fly genes were screened that were orthologous to 17 of the 20 AF-associated genes. Genes were knocked down using a heart-specific driver (Hand-Gal4 (Sellin et al., 2006)) crossed to UAS-candidate gene-RNAi lines. Progeny of the crosses were aged to three-weeks old (middle aged) and heart function was characterized. Thirteen of the genes tested exhibited significantly altered systolic intervals and/or rhythm phenotypes in the fly cardiac model and 7 of these overlapped with the genes affecting APD in the ACMs (p-value <0.001; FIG. 16). In particular, cardiac-specific KD of KNCJ5/Irk3, GATA4-6/pnr, PITX2/Ptx1, and KCNA5/Sh resulted in prolonged SIs, consistent with the increased APD observed for ACMs. Cardiac KD of KCND3/Shal and PLN/SclA significantly shortened SIs, paralleling the reductions in APD observed in ACMs. Though no significant changes in arrhythmicity were observed in the ACMs, increased arrhythmicity (AI and MAD parameters) was observed in response to cardiac KD of three genes (Irk2, Pnr, and Sk; Wilcoxcon-ranked sum test, p-values <0.001) in Drosophila.

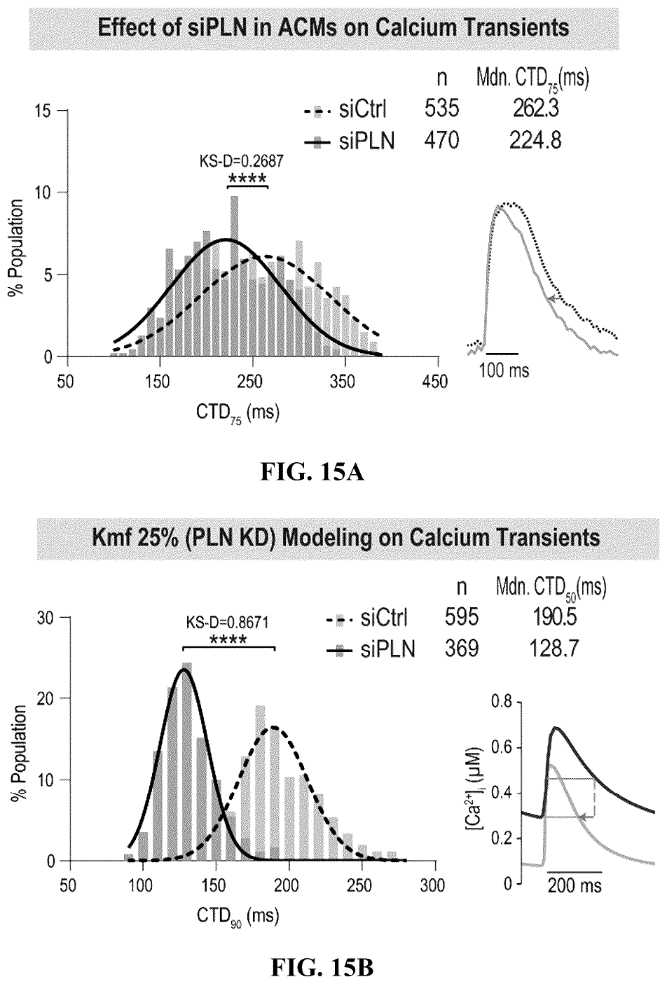

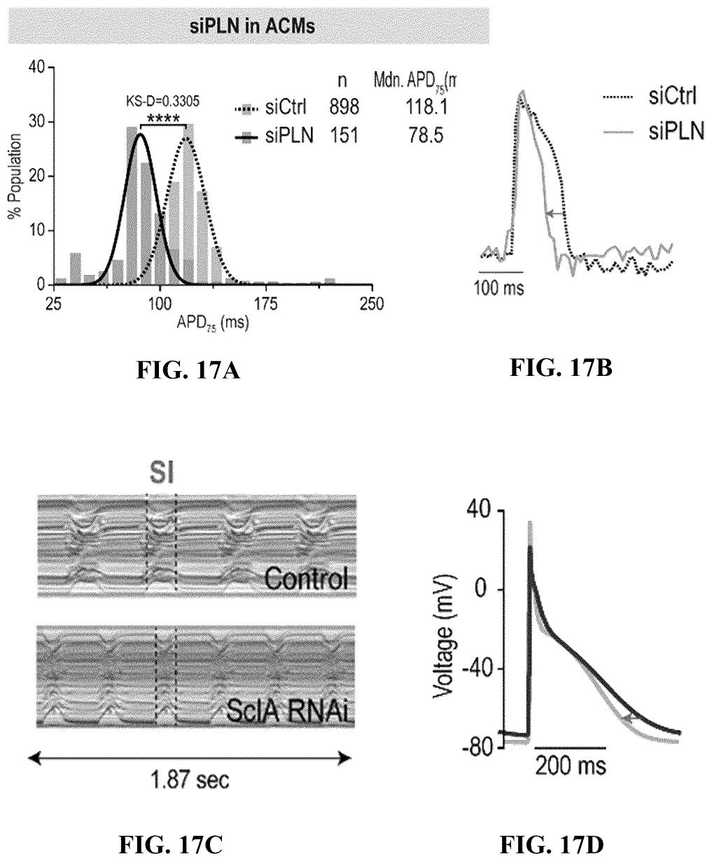

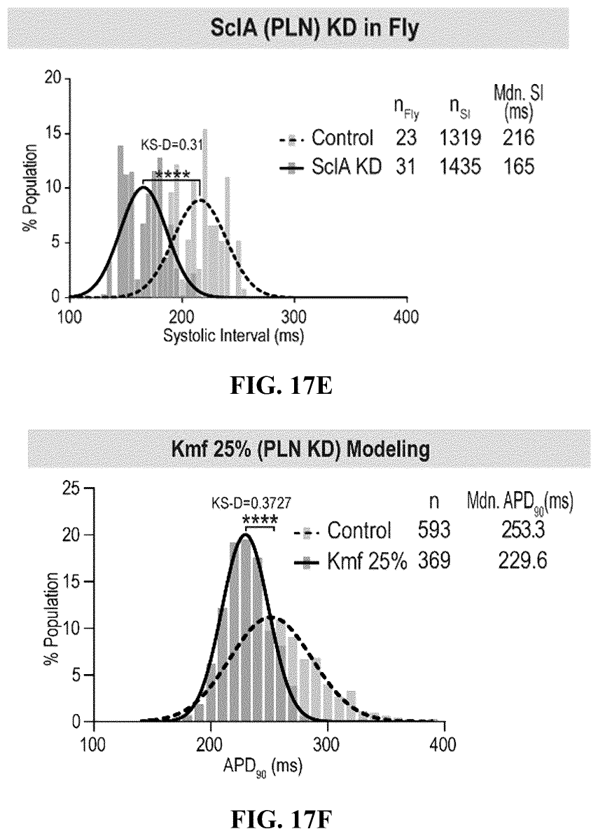

Although there is evidence that APD prolongation is associated with AF (Nielsen et al., 2013; Olson et al., 2006), APD shortening is thought to be the most common mechanism underlying the onset and maintenance of AF (Teh et al., 2012; Wakili et al., 2011). The functional screen of the present disclosure therefore focused on the gene KD that induced the strongest APD shortening phenotype. Remarkably, in both ACM and fly heart platforms, reduced PLN/SclA function consistently led to the strongest APD and SI shortening phenotype. In ACMs, PLN KD caused a significant shortening of median APD₇₅ values, from 118.1 ms to 78.5 ms (˜−40 ms) (KS-D=0.3305, p-value <0.0001), and shortened calcium transient duration (FIG. 15A, FIGS. 17A-D). Similarly, cardiac-specific KD of SclA (the PLN homolog in fly) significantly shortened contractions of the fly heart from 216 ms to 165 ms (FIGS. 17E-F).

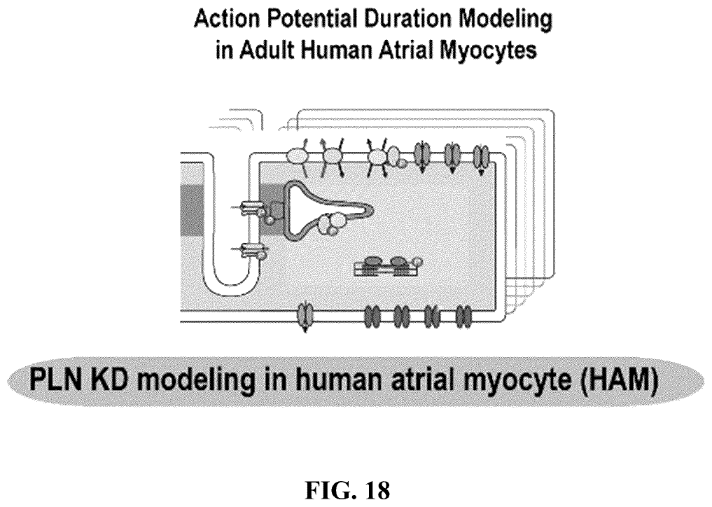

To validate the phenotypic platform findings, the functional screen of the present disclosure employed a computational modeling approach of adult human atrial myocytes (HAMs) (FIG. 18) (Grandi et al., 2011). Briefly, a population of 600 HAMs was generated with randomly varying model parameters to mimic natural cell-to-cell heterogeneity observed in cardiac tissues (Morotti et al., 2017; Ni et al., 2018) and simulated PLN KD by increasing the affinity of the sarco/endoplasmic reticulum Ca2+-ATPase (SERCA) for cytosolic Ca2+. Remarkably, and consistent with the phenotypic platform findings, computational modeling revealed that PLN KD (Kmf25%=75% reduction in PLN) caused a significant shortening of both APD90 and Ca2+ transient (FIG. 15B and FIGS. 17E-F) in HAMs as compared to control groups paced at 2 Hz. In conclusion, our multi-model system approach identifies PLN loss of function as conserved and most potent hit driving APD shortening among the 20 AF-associated genes tested.

FIGS. 14-15 show a selection of AF-associated genes for phenotypical characterization using the novel HT platform of the present disclosure. FIG. 14 shows a histogram representing the expression level (RPKM) of previously known AF-associated genes using RNAseq data from Day 12 and Day 25 ACMs. FIG. 15A shows a histogram showing the distribution of calcium transient duration (CTD) values in siControl and siPLN conditions in ACMs (right) and representative single calcium transients for both conditions (left). FIG. 15B shows a histogram of CTD generated from HAMs (left) and representative traces (right) in response to Kmf25% condition (=PLN KD). P-value ***<0.0001.

Table 3 below shows a selection of 20 AF-genes harboring a rare variant in familial AF studies and/or SNPs reported in GWAS studies.

TABLE 3
AF-Associated Gene Candidate Table

| Rare | GWAS | | |
|---|---|---|---|
| variant | Locus | Ion Channels | |
| X | X | HCN4 | Pacemaker current; I_{f} |
| X | | KCNA5 | Voltage-gated K⁺ channel; Kv1.5; I_{Kur} |
| X | X | KCND3 | Voltage-gated K⁺ channel; Kv4.3; Iₜₒ |
| X | X | KCNJ5 | Inwardly rectifying K⁺; Kir3.4; I_{KAch} |
| X | X | KCNN3 | Small conductance, Ca²⁺-activated |
| | | | K⁺ channel; KCa2.2; I_{KCa} |
| | | Transcription Factors | |
| X | X | GATA4 | GATA-binding protein 4 |
| X | X | GATA5 | GATA-binding protein 5 |
| X | X | GATA6 | GATA-binding protein 6 |
| X | X | HAND2 | Heart- and neural crest derivatives- |
| | | | expressed protein 2 |
| X | X | NKX2-5 | Homeobox protein NKX2-5 |
| X | X | NKX2-6 | Homeobox protein NKX2-6 |
| X | X | PITX2 | Paired-like homeodomain protein 2 |
| X | X | TBX5 | T-box protein 5 |
| X | X | ZFHX3 | Zinc finger homeobox protein 3 |
| | | Myocardial Structural Components | |
| X | X | GJA1 | Gap junction protein, connexin43 |
| X | X | GJA5 | Gap junction protein, connexin40 |
| X | X | SYNE2 | Nuclear envelope protein, |
| | | | spectrin repeat containing |
| | | | nuclear envelope protein 2 |
| X | X | PLN | Gap junction protein, connexin43 |
| | | Signaling | |
| X | | NPPA | Natriuretic peptide precursor A |
| X | X | SH3PXD2A | Tyrosine kinase substrate, |
| | | | SH3 and PX domains 2 |

Table 4 below shows numerical values of data presented in a heat map in FIG. 16. APD₇₅ and Systolic Intervals were normalized to their respective controls. In flies there is no homolog for NPPA, and tinman/Nkx2.5 KD flies fail to develop hearts.

TABLE 4
Effect of AFib-Associated Genes on APD

| | Normalized | Normalized |
|---|---|---|
| Gene | APD75 | Systolic Interval |
| siGATA5 | 1.22 | 1.09 |
| siGATA6 | 1.18 | 0.98 |
| siPITX2 | 1.17 | 1.09 |
| siKCNA5 | 1.16 | 1.4 |
| siGATA4 | 1.14 | 1.12 |
| siKCNJ5 | 1.14 | 1.26 |
| siHCN4 | 1.11 | 1.08 |
| siGJA1 | 1.07 | 1.18 |
| siTBX5 | 1.06 | 1.28 |
| siSYNE2 | 1.06 | 0.82 |
| siNKX2-6 | 1.04 | 1.16 |
| siSH3PXD2A | 1.02 | 0.99 |
| siKCNN3 | 0.99 | 1.3 |
| siNPPA | 0.98 | X |
| siZFHX3 | 0.98 | 1.14 |
| siNKX2-5 | 0.97 | X |
| siHAND2 | 0.96 | 1.33 |
| siGJA5 | 0.95 | 1.18 |
| siKCND3 | 0.9 | 0.82 |
| siPLN | 0.87 | 0.77 |

FIGS. 16-18 show a loss of function screen of AF-associated genes that identified conserved modulators of APD and SI in the multi-model system platform of the present disclosure. FIG. 16 shows a heatmap showing the normalized effects of AF-associated genes loss of function on APD and SI in ACMs and flies, respectively. FIGS. 17A-B shows a population distribution of APD75 values for siControl and siPLN transfected ACMs (FIG. 17A) and representative AP traces (FIG. 17B) showing the shortening effect of siPLN. FIG. 17C shows representative m-modes showing the SI shortening effect for SclA KD. FIG. 17D shows representative AP traces in HAMs showing the shortening effect on APD of simulated PLN KD. FIG. 17E shows a population distribution of SIs in control vs SclA KD conditions in flies. FIG. 17F shows a population distribution of APD90 values for Control and Kmf 25% (=PLN KD) in HAMs. FIG. 18 shows a schematic representation of APD modeling in HAMs.

Table 5 below shows screen results in ACMs. The table displays median, normalized median, KS-D, and P-values related to FIG. 14.

TABLE 5

| | Median | Norm Median | | P-value of |
|---|---|---|---|---|
| Gene ID | APD75 | APD75 | KSD | KS-D |
| Ctrl | 133.6 | 1 | | |
| GATA4 | 151.8 | 1.136227545 | 0.1577 | <0.0001 |
| GATA5 | 162.6 | 1.217065868 | 0.2691 | <0.0001 |
| GATA6 | 157.8 | 1.181137725 | 0.2482 | <0.0001 |
| GJA1 | 142.5 | 1.066616766 | 0.05343 | 0.0019 |
| GJA5 | 127.4 | 0.953592814 | 0.2149 | <0.0001 |
| HAND2 | 127.6 | 0.95508982 | 0.2466 | <0.0001 |
| HCN4 | 148.4 | 1.110778443 | 0.1756 | <0.0001 |
| KCNA5 | 155.5 | 1.163922156 | 0.2014 | <0.0001 |
| KCND3 | 119.6 | 0.895209581 | 0.2371 | <0.0001 |
| KCNJ5 | 152.4 | 1.140718563 | 0.1893 | <0.0001 |
| KCNN3 | 132.4 | 0.991017964 | 0.1503 | <0.0001 |
| NKX2-5 | 129.9 | 0.972305389 | 0.1762 | <0.0001 |
| NKX2-6 | 138.3 | 1.035179641 | 0.04106 | 0.0275 |
| NPPA | 131.4 | 0.983532934 | 0.1047 | <0.0001 |
| PITX2 | 156 | 1.167664671 | 0.2053 | <0.0001 |
| PLN | 116.1 | 0.869011976 | 0.3305 | <0.0001 |

Table 6 below shows screen results in flies. The table displays normalized systolic interval (SI), standard deviations, and P values related to FIG. 14.

TABLE 6

| Fly gene | Norm SI | Deviations | P-Value |
|---|---|---|---|
| Sh | 1.40333138 | 0.40333138 | 6.91^{E}-10 |
| Hand | 1.32601473 | 0.32601473 | 2.52^{E}-09 |
| SclA | 0.77328487 | 0.22671513 | 4.93^{E}-09 |
| Sk | 1.25795515 | 0.25795515 | 4.99^{E}-08 |
| Doc1 | 1.26981919 | 0.26981919 | 4.57^{E}-07 |
| Pnr | 1.12001245 | 0.12001245 | 5.21^{E}-06 |
| Shal | 0.81835564 | 0.18164436 | 1.31^{E}-05 |
| Irk3 | 1.30245197 | 0.30245197 | 1.55^{E}-05 |
| shakb | 1.15642458 | 0.15642458 | 1.18^{E}-04 |
| Scro | 1.16119611 | 0.16119611 | 0.00021027 |
| MSP300 | 0.81943812 | 0.18056188 | 0.00046931 |
| Ih | 1.07869321 | 0.07869321 | 0.01360059 |
| Ptx 1 | 1.08533093 | 0.08533093 | 0.0543814 |
| Zfh-2 | 1.14018008 | 0.14018008 | 0.08787157 |
| Cindr | 0.9867872 | 0.0132128 | 0.71808748 |

### Example 4: PLN Loss of Function-Induced Arrhythmia Depends on β-Adrenergic Pathway Stimulation and Co-Culture with Fibroblasts

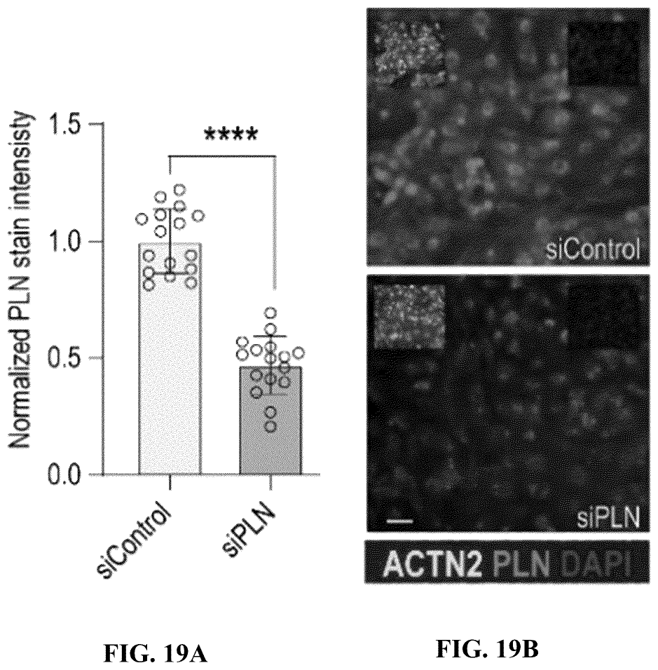

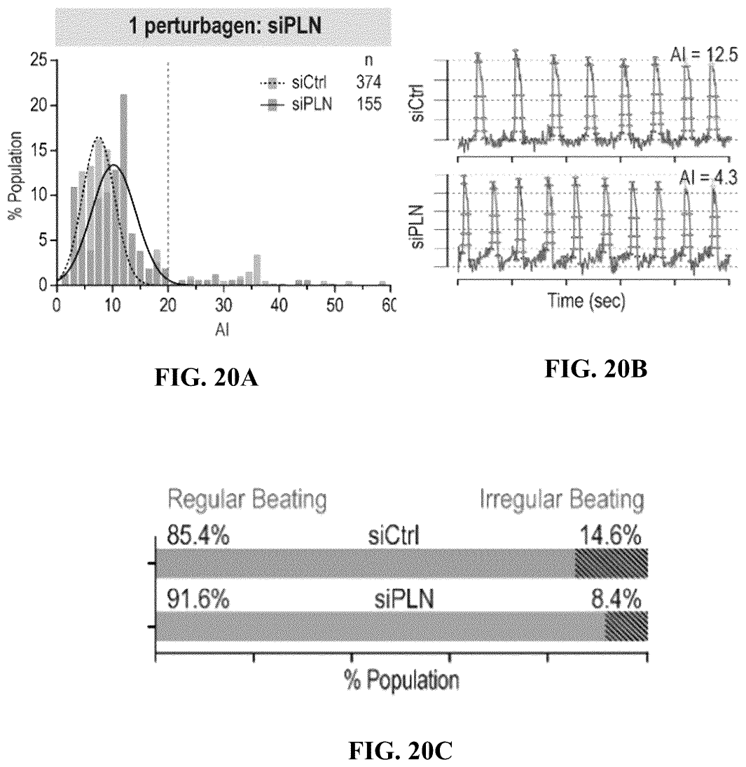

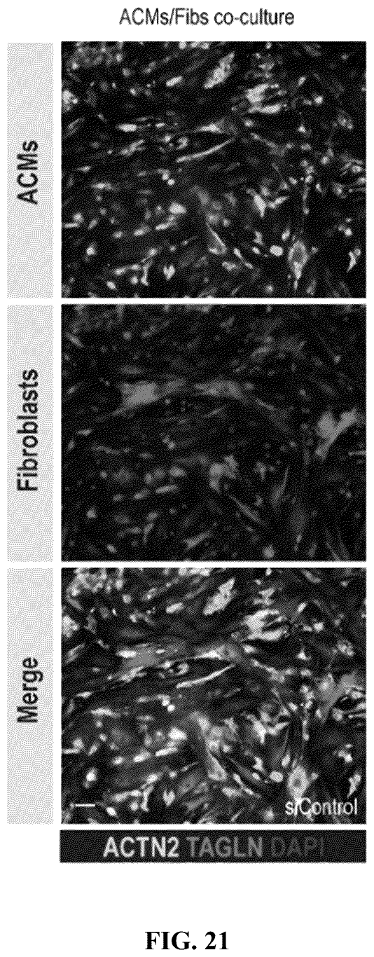

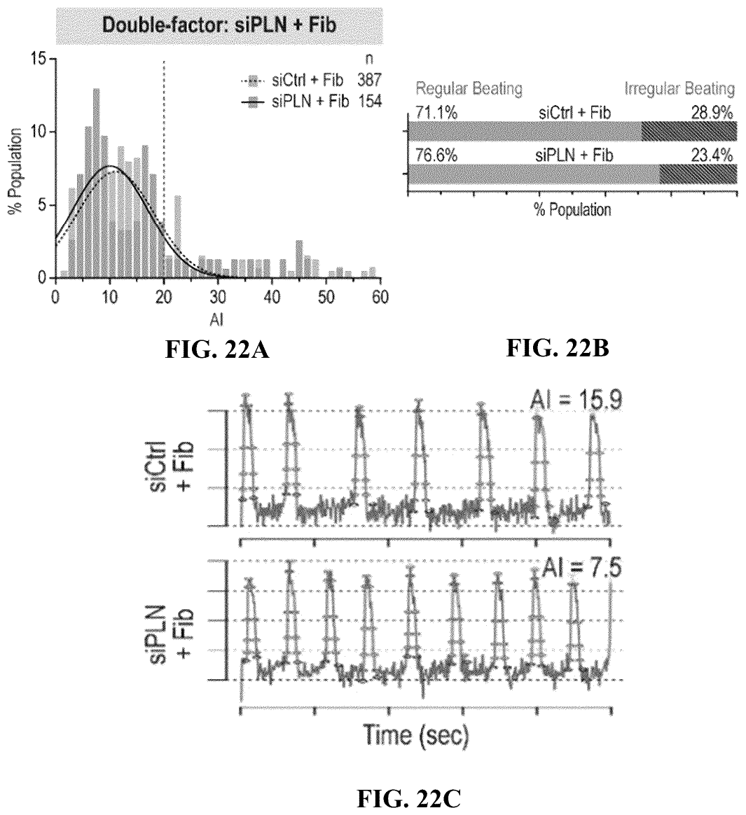

In this example, the effects of PLN knockdown were assessed in ACMs and flies. To determine if loss of PLN function alone is sufficient to induce arrhythmia-like phenotypes, the beat-to-beat interval variance (arrhythmia index, AI) was measured in ACMs upon PLN KD, and no difference was found as compared to siControl (FIGS. 19A-B and FIGS. 20A-C). Additional factors such as conductance heterogeneity due to atrial fibrosis (Dzeshka et al., 2015; Xintarakou et al., 2020) as well as sympathetic stresses (Workman, 2010) have been linked with the onset and maintenance of AF and are often categorized as “AF-associated risk factors”. Thus, to mimic these environmental perturbagens, ACMs were co-cultured with fibroblasts for two days and/or the b-adrenoreceptor agonist, isoproterenol (1 mM), was acutely applied upon kinetic imaging. Remarkably, co-culturing ACMs with fibroblasts in a 3:1 ratio nearly doubled the percentage of ACMs with AI values >20 (from 14.6% to 28.9%), however, in this context, PLN KD did not further increase the percentage of arrhythmic cells (FIGS. 20B-C, FIG. 21, and FIGS. 22A-C).

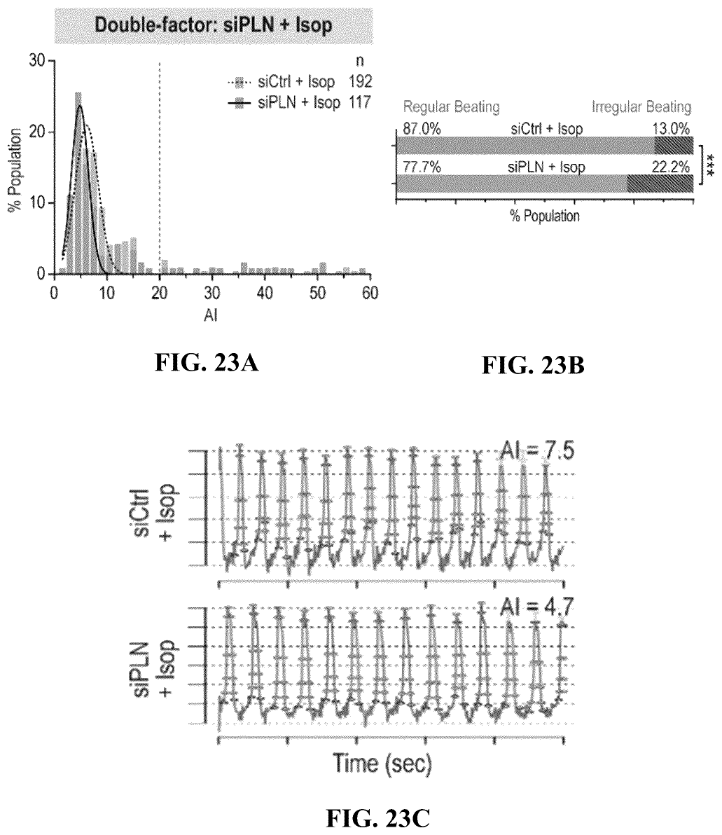

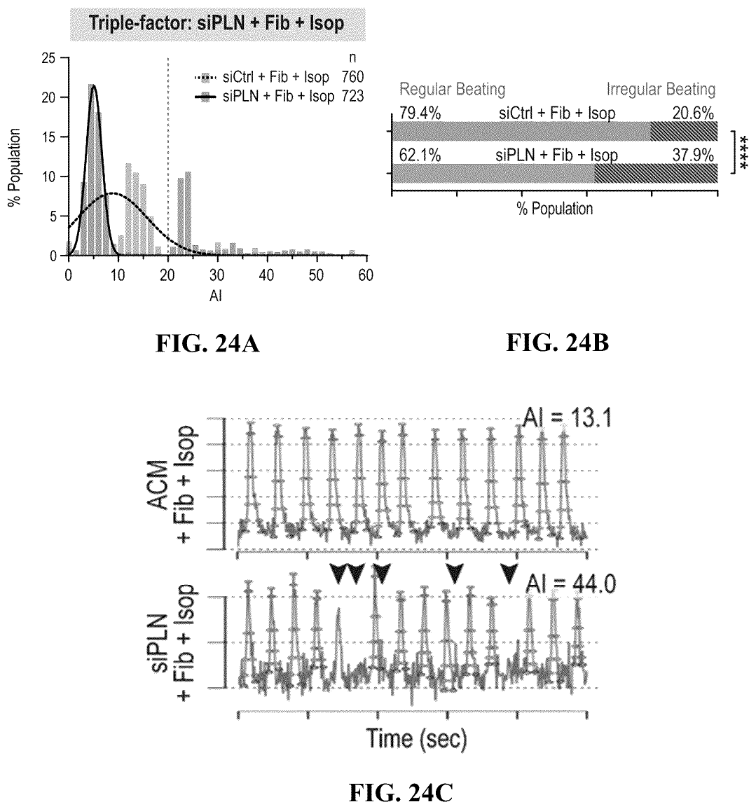

Conversely, treating ACMs with Isoproterenol alone did not increase the percentage of arrhythmic cells, while exposing ACMs to Isoproterenol along with PLN KD, increased the percentage of arrhythmic cells from 13% to 22% (FIGS. 23A-C). Finally, co-culture with fibroblasts followed by acute isoproterenol treatment caused severe arrhythmia-like phenotypes (AI values >20) in ˜ 38% of ACMs as compared to 20% in perturbagens only (FIGS. 24A-C). Interestingly, analysis of AP trains (lower panel of FIG. 24C) revealed missing beats and smaller refiring events that could be associated with delayed afterdepolarizations (DADs). Thus, collectively the results of the present disclosure indicate that reduced PLN function predisposes ACMs to arrhythmia upon sensitization by fibroblasts and acute β-adrenergic stimulation.

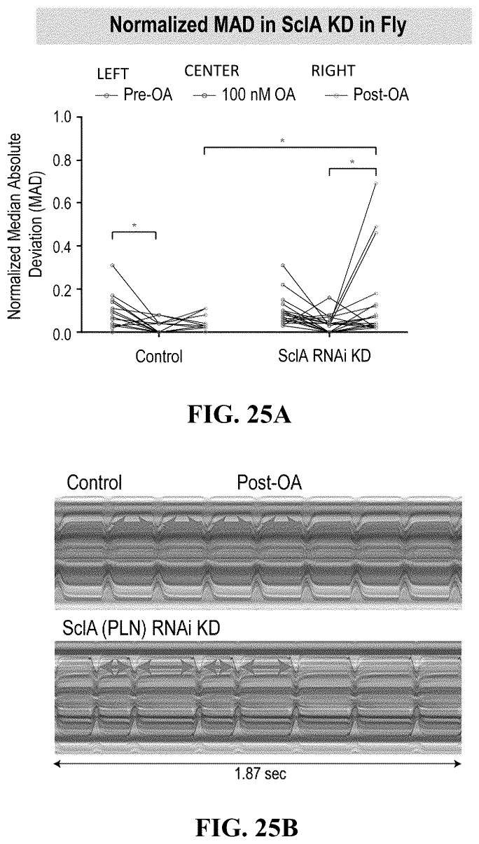

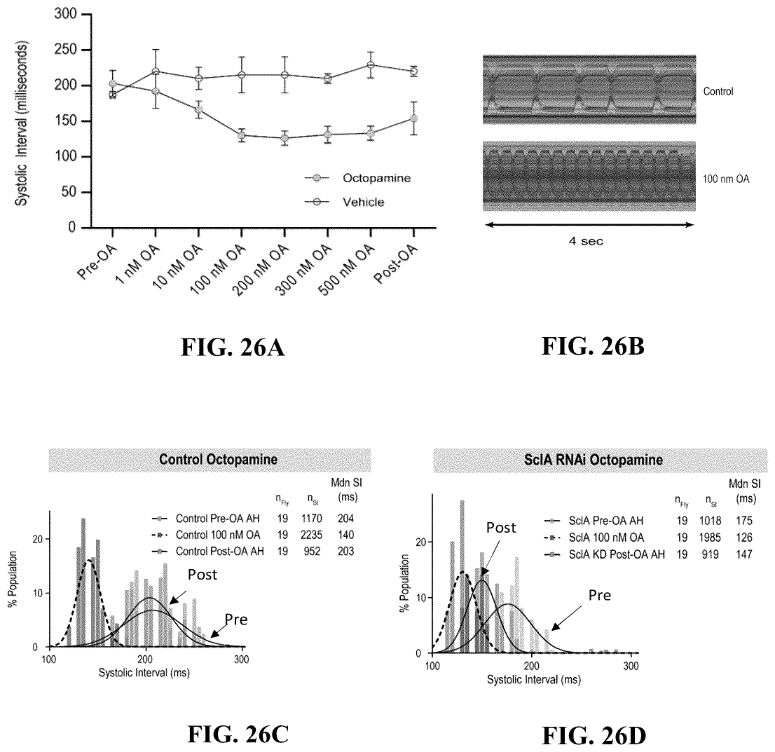

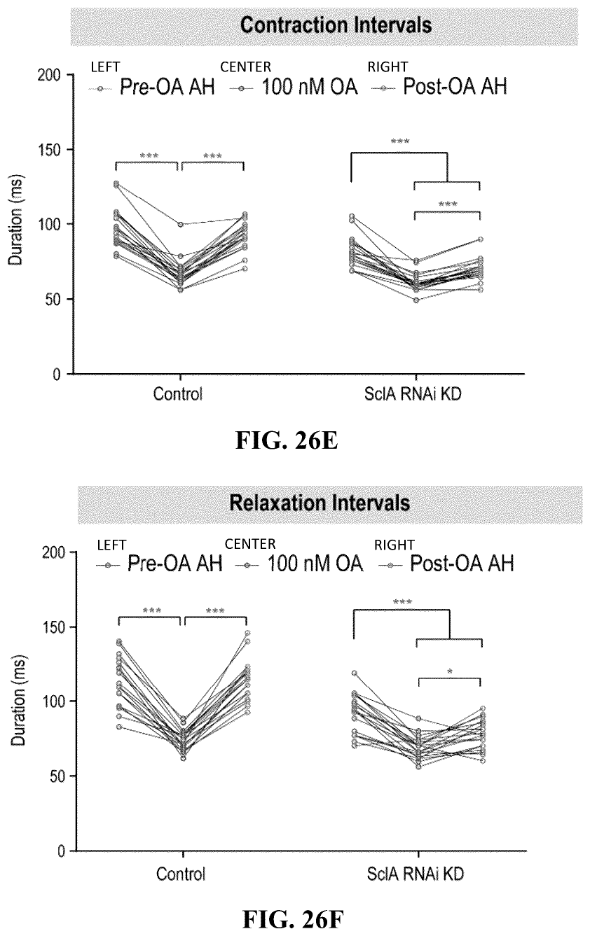

In fly hearts, despite significant changes in SI, neither AI nor MAD arrhythmia parameters were significantly altered by cardiac-specific PLN/SclA KD. To add an adrenergic stress, the fly hearts were exposed to octopamine (OA), the fruit fly version of norepinephrine/adrenaline (Sujkowski et al., 2017). Acute OA exposure significantly elevated heart rate by significantly shortening systolic intervals in both control and KD lines with a maximal effect at 100 nM, which was the dose used for subsequent pharmacological pacing of the fly heart (FIGS. 26A-B). In controls, the mean SI returned to pre-exposure values at 10 min post-OA exposure (FIG. 26C), whereas the PLN/SIn KD hearts did not (FIG. 26D). In addition to SI, both contraction and relaxation intervals were significantly shortened in the presence of OA (FIGS. 26E-F). Increased post-octopamine pacing bouts of arrhythmia were also observed in the PLN/SclA KD hearts (mean nMAD=0.1342; FIGS. 25A-B) as compared to hearts from controls (mean nMAD=0.0379; p-value: 0.019, repeated measures two-way ANOVA).

FIGS. 19-24 show the testing of multiple perturbagens to trigger arrhythmia in ACMs. FIG. 19A shows a histogram showing the quantification of PLN staining intensity in siControl and siPLN conditions in ACMs. FIG. 19B shows representative images of ACMs stained for actinin2 (ACTN2) to mark ACMs and for phospholamban (PLN) showing a significant reduction of PLN protein levels upon PLN KD. FIG. 20A shows a histogram showing the distribution of AI values from ACMs in siControl and siPLN conditions. FIG. 20B shows representative AP traces in siControl and siPLN conditions. FIG. 20C shows quantification of irregular AP peak trains. FIG. 21 shows representative images of ACMs co-cultured with human dermal fibroblasts and stained with ACTN2 (cardiac, top panel) and TAGLN (fibroblasts, middle panel). FIG. 22A shows a histogram showing the distribution of AI values from ACMs co-cultured with fibroblasts (Fib) in siControl and siPLN conditions. FIG. 22B shows quantification of the percentage of ACMs with irregular AP peak trains for each condition. FIG. 22C shows representative AP peak trains for each condition. FIG. 23A shows a histogram showing the distribution of AI values from ACMs treated with Isoproterenol in siControl and siPLN conditions. FIG. 23B shows quantification of the percentage of ACMs with irregular AP peak trains for each condition. FIG. 23C shows representative AP peak trains for each condition.

FIGS. 24-26 shows that PLN KD induces arrhythmia phenotypes in combination with environmental pertubagens in both ACMs and flies. FIG. 24A shows a population distribution of arrhythmia index (AI) values of ACMs co-cultured with fibroblasts and treated with isoproterenol (Isop) in siControl vs siPLN conditions. FIG. 24B shows histograms showing the increased percentage of irregularly beating (AI>20) of ACMs co-cultured with fibroblasts and treated with Isop, in siPLN as compared to siControl. FIG. 24C shows representative peak trains of APs showing irregular beat to beat intervals (black arrowheads) in siPLN as compared to siControl condition. FIG. 25A shows the distribution of Median Absolute Deviation (MAD) values before (left data points), during (center data points) and after 100 nM octopamine treatment (OA) (right data points). Post-OA, SclA KD hearts exhibit increased arrhythmia as compared to controls (p-value <0.05, repeated measures 2-way ANOVA). FIG. 25B shows representative M-modes showing irregular beat to beat intervals in SclA KD hearts post-OA as compared to control (arrows show individual heart periods).

FIGS. 26A-F show the optimization of octopamine treatment in flies. FIG. 26A shows a graph showing average SI values in response to escalating doses of OA in flies. FIG. 26B shows representative M-modes from one heart before (top) and during application of 100 nM OA (bottom) showing dramatic increases in heart rate. FIG. 26C shows histograms showing the distribution of SI values in control hearts pre-OA pacing, during exposure to 100 nM OA, and 15 min post-OA application. Distribution of SIs post-OA returns to that of pre-OA. FIG. 26D shows histograms showing the distribution of SI values in SclA KD hearts pre-OA pacing, during exposure to 100 nM OA, and 15 min post-OA application. Distribution of SIs post-OA are significantly shorter compared to pre-OA and are due to decreases in both the contraction (FIG. 26E) and relaxation (FIG. 26F) phases of the systolic intervals (p-value <0.05, repeated measures 2-way ANOVA).

### Example 5: Computational Modeling Validates PLN as a Key Regulator of Rhythm in Human Adult Atria

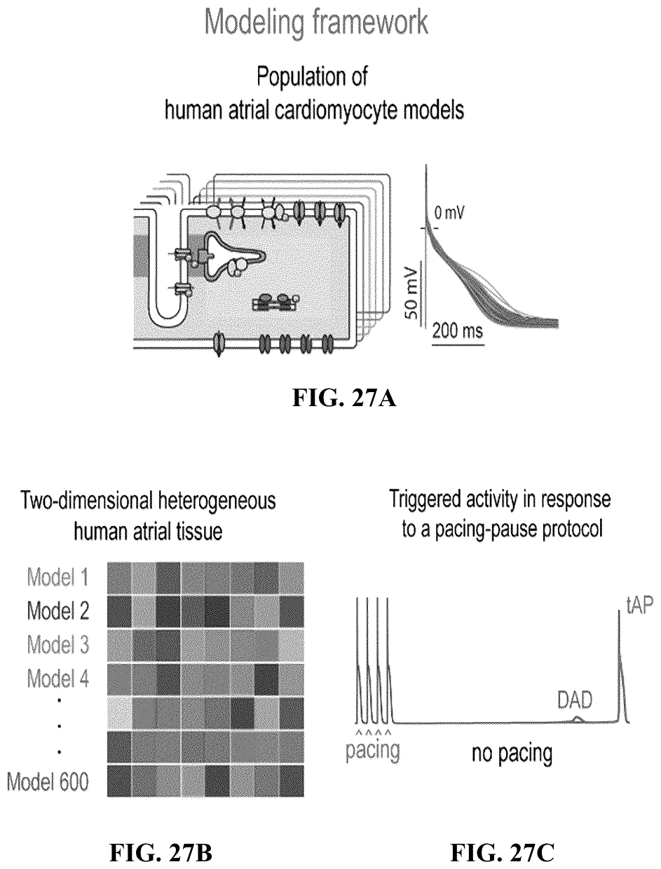

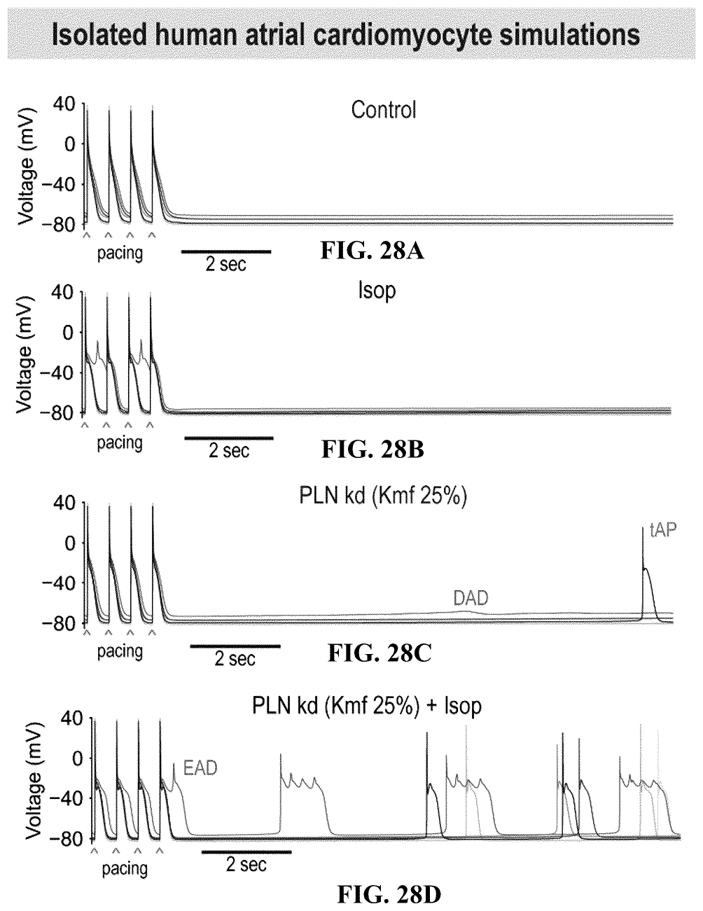

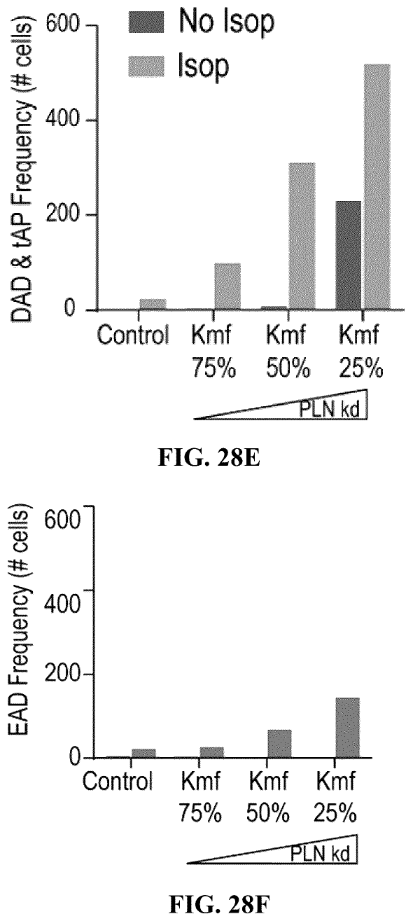

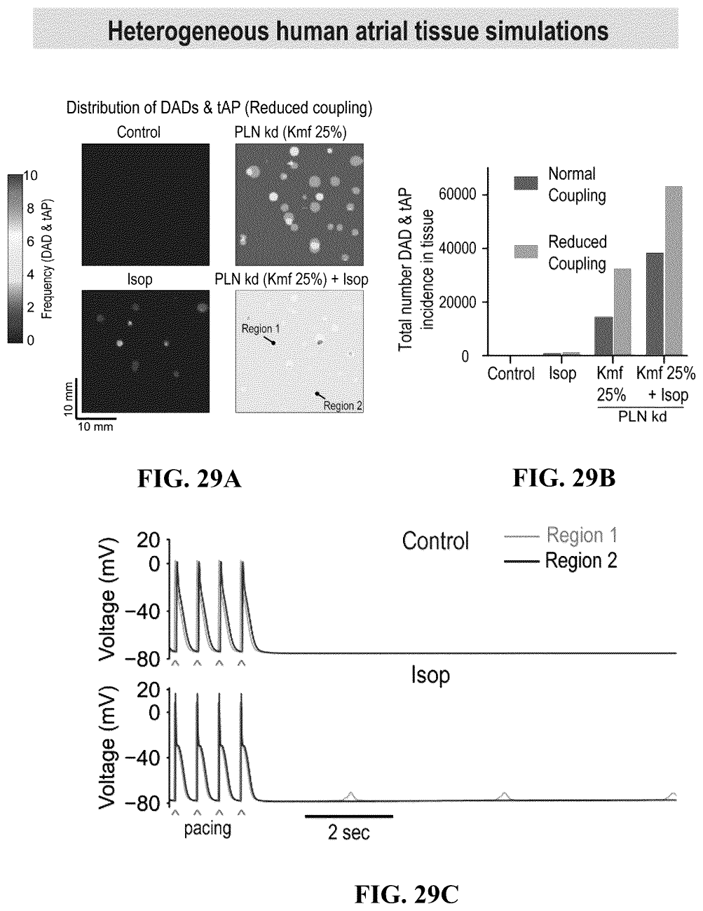

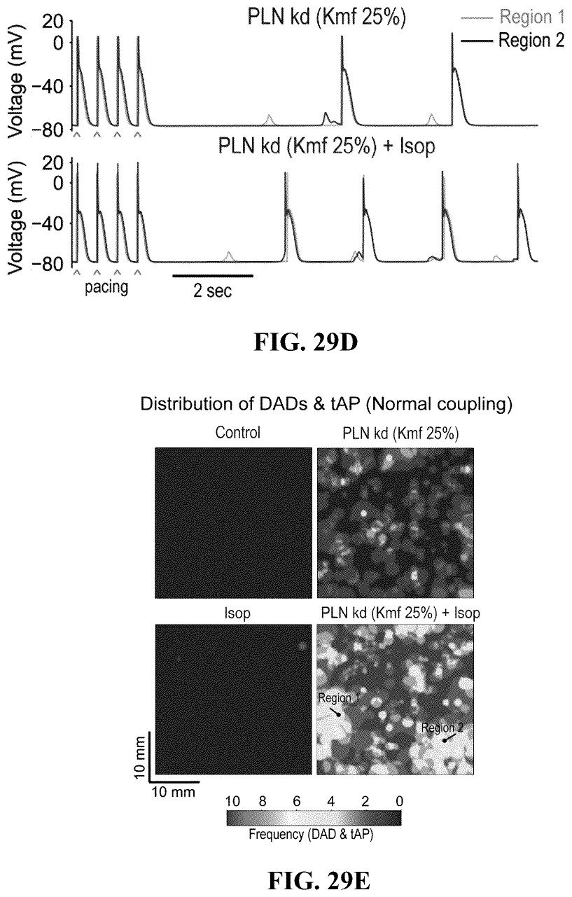

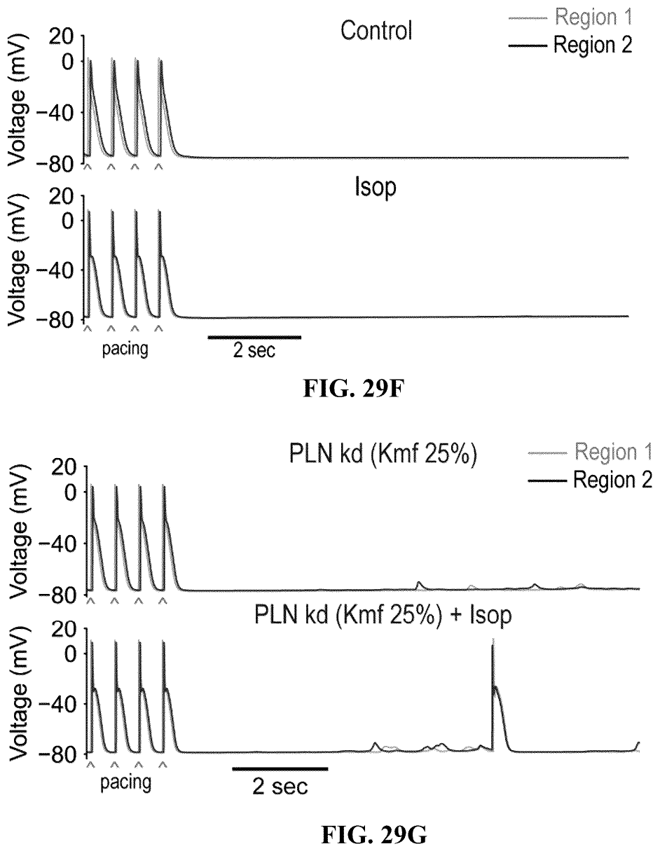

In this example, computational modeling was used to validate PLN as a key regulator of rhythm in human adult atria. To validate the phenotypic platform findings, models were used of both isolated HAMs and two-dimensional atrial tissue that allows to modulate cell-cell electrical coupling (FIGS. 27A-C, Table 7, and Colman et al., 2013; Ni et al., 2018), that accounts for electronic effects of fibroblasts. In these assays, a 2-Hz pacing-pause protocol was applied to stimulate isolated HAMs or the left side of the atrial tissue construct and subsequently membrane voltage dynamics were analyzed following the pause of pacing. First, the effect of increasing PLN KD on isolated HAMs was tested (Kmf 25%=high KD, Kmf 75%=low KD) along with simulated isoproterenol treatment. Remarkably, increasing PLN KD levels led to the enhanced generation of triggered activity, which was further exacerbated by the simulated isoproterenol treatment (FIGS. 28A-F). In this context, increasing PLN KD levels in combination with simulated isoproterenol treatment also led to an increase in occurrence of early afterdepolarizations (EADs) in isolated HAMs (FIG. 28F). At the tissue level and similar to fibroblast co-culture with ACMs, PLN KD induced more triggered activity (DADs and triggered APs) when combined with treatment with isoproterenol and reduced cell-cell electrical coupling (FIGS. 29A-G). Collectively, the findings of the present disclosure suggest that PLN loss of function predisposes cells to arrhythmia in a tissue environment with reduced electrical coupling and elevated b-adrenergic activity.

FIGS. 27-29 show that a combined PLN KD and Isop challenge promoted arrhythmic events in both isolated human atrial myocytes (HAMs) and two-dimensional atrial constructs. FIG. 27A shows a modeling framework for evaluating arrhythmic events in HAMs and two-dimensional (2D) human atrial tissue as well as an example graph depicting action potentials generated by this model. Shades of gray in FIG. 27B indicate that each cell from the population of computational models has distinct electrophysiological properties (i.e., AP waveforms) to mimic physiologic heterogeneity in cells. For the 2D model of human atrial tissue, each of the 600 cells was mapped into clusters, each of which has distinct properties compared to neighboring clusters. The physiological properties of each myocyte cluster were randomly assigned, thereby producing a heterogeneous tissue structure. A pacing (2 Hz)-pause protocol was applied to assess the incidence of triggered activities (FIG. 27C). FIGS. 28A-D shows the effects of PLN KD (Kmf 25%) and Isop on the triggered activity in human atrial cardiomyocytes: FIG. 28A shows time courses of APs for baseline (control), with Isop treatment (FIG. 28B), PLN KD (=Kmf 25%) (FIG. 28C), and combined Isop treatment and PLN KD (PLN KD+Isop) (FIG. 28D). FIGS. 28E-F shows the incidence of DAD and tAP (FIG. 28E) and EAD (FIG. 28F) detected in the HAM populations for Isop and various degrees of PLN KD (Kmf varied from 25% to 75%) conditions. FIGS. 29A-G shows the effects of PLN KD (Kmf 25%) and Isop on the generation of triggered activity in heterogeneous human atrial tissue: FIG. 29A shows the spatial distribution of DADs and tAPs in the atrial tissue with reduced cell-to-cell coupling for PLN KD (Kmf 25%) and after Isop treatment. FIG. 29B shows the total number of DADs and tAPs detected in the atrial tissue after each perturbation with normal or reduced cell-to-cell coupling. FIG. 29C shows the superimposed traces of APs from two regions (marked in FIG. 29A) of the atrial tissue with reduced cell-to-cell coupling for each perturbation. FIG. 29D show the effects of PLN kd (Kmf 25%) and Isop on the triggered activity in heterogeneous human atrial tissue with normal cell-to-cell coupling. Tissue was paced at 2 Hz for 10 s followed by a period of 10 s without stimulation. FIG. 29E shows the spatial distribution of DADs and tAPs in the atrial tissue for PLN KD (Kmf 25%) and after Isop treatment. FIGS. 29F-G shows the superimposed traces of APs from two regions (marked in FIG. 29E) of the atrial tissue with normal cell-to-cell coupling for each perturbation.

Table 7 below shows conduction velocity measured in 2D tissue.

TABLE 7

| | Conduction velocity | |
|---|---|---|
| Pacing rate | 1 Hz | 2 Hz |
| Normal coupling | 0.63 m/s | 0.41 m/s |
| (D × 100%) | | |
| Reduced coupling | 0.28 m/s | 0.19 m/s |
| (D × 25%) | | |

### Example 6: PLN Functionally Interacts with NCX and L-Type Calcium Channels to Control Rhythm

In this example, regression analysis and genetic perturbations were conducted to further characterize the role of PLN in the regulation of atrial rhythm.

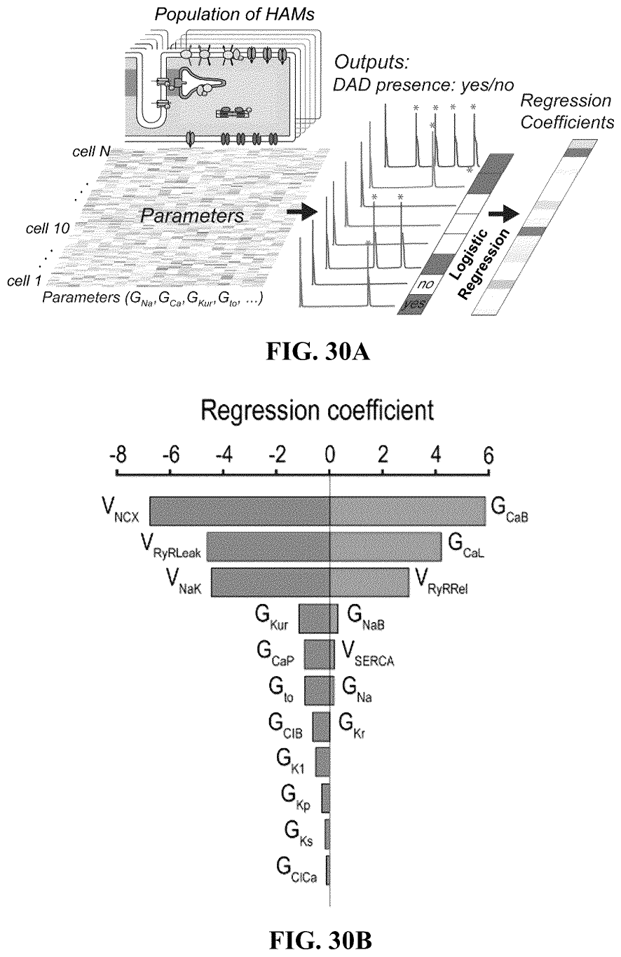

To characterize how PLN control rhythm homeostasis in atrial myocytes, it was noted by the inventors that at Kmf 50%, only half of HAMs generated DADs (see FIG. 28E). Thus, to uncover mechanisms underlying DAD-generation in HAMs at Kmf 50%, DAD-generating HAMs were separated from non-DAD-generating, and logistic regression analysis was applied (Morotti and Grandi, 2017) to describe the relationship between model parameters and DAD incidence (FIG. 30A and Table 8). This analysis predicted that increased Ca²⁺ current I_{CaB}, L-type Ca²⁺ current I_{CaL}, or RyR release flux (i.e., by augmenting the parameters G_{CaB}, G_{CaL} or V_{RrRRel}) in HAMs would promote the propensity for developing DADs. Similarly, the model of the present disclosure also predicted that DAD occurrence correlated with reduced function of sodium-calcium exchanger NCX, RyR leakiness, sodium-potassium pump NaK, or ultra-rapid delayed rectifier K+ current I_{Kur} (FIG. 30B).

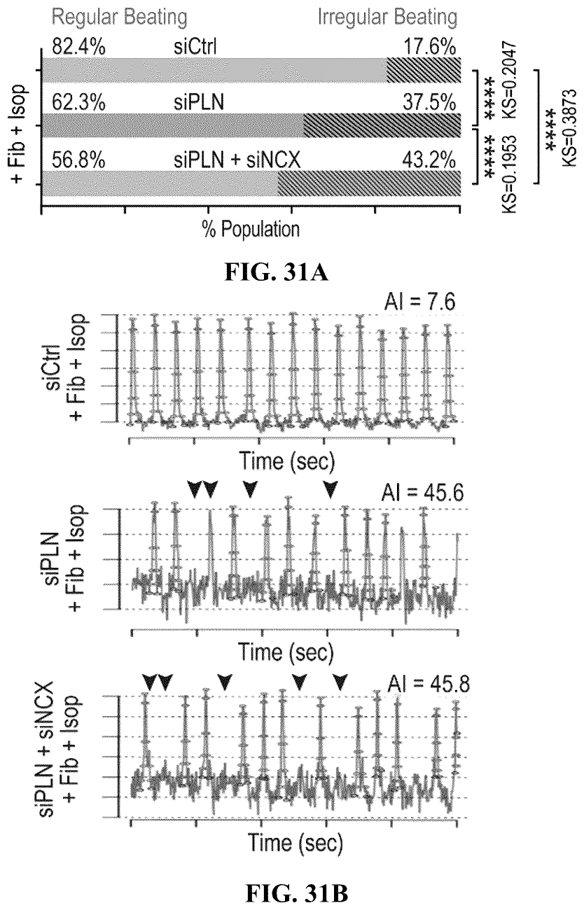

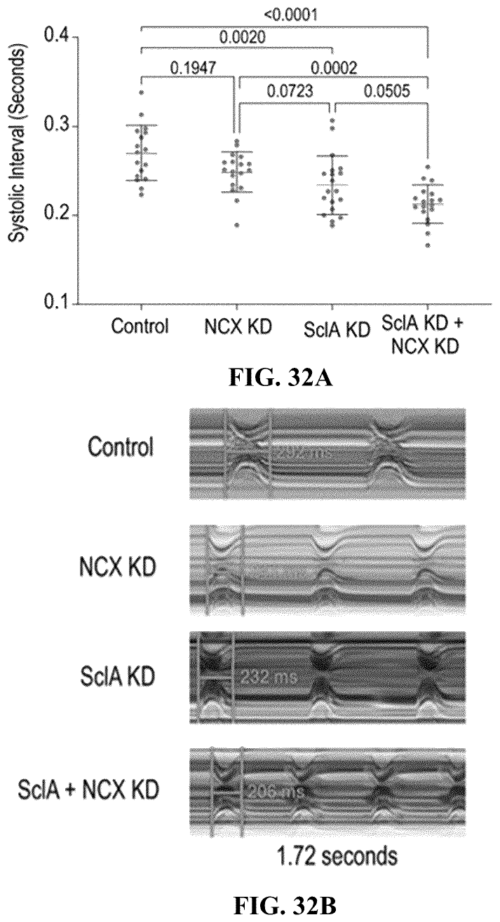

To validate these predictions, two parameters were selected that were most positively (ICaL conductance) or negatively (NCX maximal transport rate) correlated with DAD incidence. First, in ACMs it was tested whether reduced expression of the sodium-calcium exchanger NCX in the background of PLN KD would further increase the percentage arrhythmic cells. Consistent with the model prediction, combined KD of PLN and NCX in the presence of perturbagens (fibroblasts co-culture and isoproterenol infusion) significantly increased the occurrence of arrhythmia-like phenotypes in ACMs as compared to single PLN KD, from 37.5% to 43.2% of cells with AI>20 (FIG. 31A). Notably, the increased arrhythmic phenotypes observed in siPLN/NCX treated ACMs was accompanied by short APs as compared to siPLN alone (FIG. 31B). To determine whether the interaction between PLN and NCX is conserved at the whole heart level, single KD and co-KD of NCX/Calx and PLN/SclA were generated using the Hand4.2-Gal4 heart-specific driver line flies. Remarkably, while median SI was reduced in response to SclA/Pln KD (232 ms) or NCX/Calx KD (253 ms, FIGS. 32A-B), KD of both genes further shortened the SI median to 211 ms (p=0.05, Wilcoxon ranked sum test).

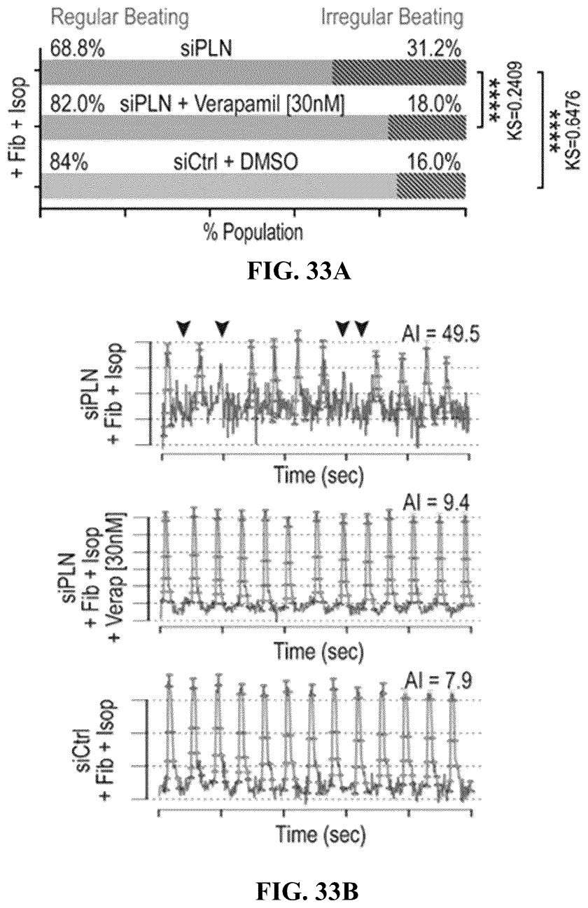

Finally, the regression analysis also revealed that DAD-generating HAMs had increased L-type calcium channel (G_{CaL}) currents. Thus, to test whether inhibition of L-type calcium channels activity might reduce PLN-induced arrhythmia phenotypes, ACMs were treated with a calcium channel blocker verapamil and the percentage of arrhythmic cells was quantified in response to PLN KD+Fib+isoproterenol treatment. Remarkably, ACMs treated with verapamil were 1.7 fold less arrhythmic than DMSO control (from 31.2% to 18%, FIGS. 33A-B). Collectively, the results of the present disclosure indicate that PLN KD-dependent arrhythmia phenotypes are at least in part mediated by NCX and L-type calcium channel activity. The results of the present disclosure also highlight that the combined use of computational modeling and the phenotypic platform represents an effective approach to identify gene interactions involved in the regulation of atrial rhythm and to potentially predict potential therapeutic targets to treat AF.

FIGS. 30-33 show that multiple perturbations are required to generate arrhythmicity across platforms. FIG. 30A shows a schematic describing the logistic regression analysis approach to identify the mechanisms underlying the generation of DADs in HAMs. FIG. 30B shows logistic regression analysis of DAD incidence in the context of moderate PLN knockdown (Kmf 50%), which revealed the influence of model parameters on the genesis of DADs in the population of HAMs in response to the pacing-pause protocol. Positive coefficients indicated that increasing the associated parameters promotes DAD production, and vice versa. FIG. 31A shows a histogram showing that the percentage of irregularly beating (AI>20) ACMs co-cultured with fibroblasts and treated with isoproterenol, is increased when transfected with siPLN and NCX as compared to siPLN alone. FIG. 31B shows representative AP peak trains for siControl, PLN siPLN+NCX conditions in ACMs co-cultured with fibroblasts and treated with isoproterenol. Arrowheads show examples of irregular beat to beat intervals in arrhythmically beating ACMs. FIG. 32A shows mean Systolic Interval (SI) in response to cardiac KD of the plasma membrane Na/Ca2 exchanger NCX, SclA, and combined SclA+NCX KD. Co-KD caused a greater decrease in SI than did single KD alone (p<0.05, Wilcoxon ranked sum test). FIG. 32B shows representative m-modes showing effects of KD on SI. FIG. 33A shows a histogram showing that the percentage of irregularly beating (AI>20) ACMs co-cultured with fibroblasts and treated with isoproterenol, is decreased when treated with verapamil (30 nM) as compared to DMSO. FIG. 33B shows representative AP peak trains and AI values for siPLN; siPLN+verapamil (30 nM); siControl conditions in ACMs co-cultured with fibroblasts and treated with Isop. Arrowheads show examples of irregular beat-to-beat intervals in arrhythmically beating ACMs. ****P<0.0001 (KS-D).

Table 8 below shows a glossary for model parameters that were perturbed for constructing populations of human atrial models.

TABLE 8

| Parameter | Note |
|---|---|
| G_{Na} | Fast Na⁺ current, maximal conductance |
| G_{CaL} | L-type Ca²⁺ current, maximal conductance |
| Gₜₒ | Transient outward K⁺ current, maximal conductance |
| G_{Kur} | Ultra-rapid delayed rectifier K⁺ current, maximal |
| | conductance |
| G_{Kr} | Rapid delayed rectifier K⁺ current, maximal conductance |
| G_{Ks} | Slow delayed rectifier K⁺ current, maximal conductance |
| G_{K1} | Inward rectifier K⁺ current, maximal conductance |
| G_{Kp} | Conductance of the plateau K⁺ current |
| G_{NaB} | Background Na⁺ current, maximal conductance |
| G_{CaB} | Background Ca²⁺ current, maximal conductance |
| G_{CaP} | Sarcoplasmic Ca²⁺ pump current, maximal pump rate |
| G_{ClCa} | Ca²⁺ activated Cl⁻ current, maximal conductance |
| G_{ClB} | Background Cl⁻ current, maximal conductance |
| V_{NCX} | Na⁺/Ca²⁺ exchange current, maximal exchange rate |
| V_{NaK} | Na⁺/K⁺ pump current, maximal pump rate |
| V_{SERCA} | Rate of the SERCA pump |
| V_{RyR,Rel} | Rate of the SR Ca²⁺ release via ryanodine receptors |
| V_{RyR,Leak} | Rate of the SR Ca²⁺ leak via ryanodine receptors |

### Example 7: The Multi-Modal HT Phenotypic Platform of the Present Disclosure is Used to Screen for Candidate Agents for the Treatment of a Cardiac Rhythm Disorder

In this example, cardiomyocytes of the present disclosure are infused with a candidate agent as described in Example 1, and an AI value is generated to describe the effect of the candidate agent on a phenotype associated with a cardiac rhythm disorder. In certain embodiments, the cardiomyocytes are ACMs of the present disclosure. In some embodiments, the cardiomyocytes are cultured with fibroblasts prior to being contacted with the candidate agent. In many embodiments, the cardiomyocytes are further labeled with a voltage dye or a nuclear dye. In many embodiments, the voltage-dependent fluorescence variation of the voltage dye is quantified over time. In some cases, action potential traces of the cardiomyocytes are automatically processed. In many cases, parameters associated with infusion of the candidate agent are measured. The parameters are selected from the group comprising: APD-10, 25, 50, 75, 90; T25-75, T75-25; Vmax up and down; beat rate; peak-to-peak interval; and rhythm regularity index. In some embodiments, the candidate agent is a nucleic acid. In other embodiments, the candidate agent is a small molecule. In certain embodiments, the candidate agent is a protein. In some cases, the nucleic acid recognizes the gene phospholamban (PLN). In many cases, the nucleic acid recognizes the gene NCX. In some embodiments, the cardiomyocytes are assessed at single cell resolution.

### Example 8: An Increased Risk for Atrial Fibrillation (AF) is Determined in a Human Subject

In this example, analyses of the present disclosure are used to determine an increased risk for AF in a human subject. In some embodiments, a biological sample is collected from a human subject. In other embodiments, a level of a gene or gene product associated with AF in the biological sample is determined by an assay of the present disclosure. In embodiments, the gene or gene product is identified using the novel multiplatform modeling approach of the present disclosure. In some cases, the assay further comprises contacting the biological sample with a reagent that recognizes the gene or gene product associated with AF. In some embodiments, the biological sample is blood from a human subject. In certain embodiments, the biological sample is a DNA sample. In some cases, the assay comprises genomic sequencing of the human subject. In certain cases, the assay comprises a proteomic assay. In certain embodiments, a therapeutic agent is administered to the human subject. In some cases, the therapeutic agent is configured to alleviate or mitigate one or more symptoms of AF in the human subject. In some embodiments, the gene is PLN. In other embodiments, the gene is NCX. In some cases, the gene product is an L-type Calcium channel. In certain embodiments, the therapeutic agent is verapamil.

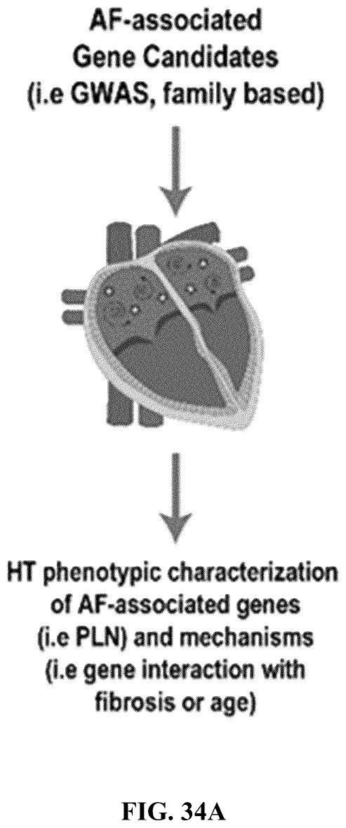

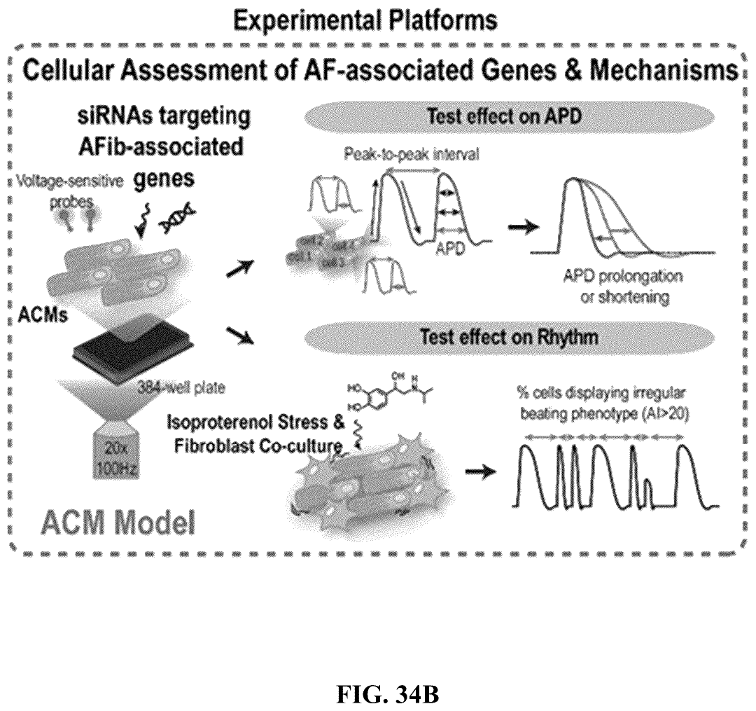

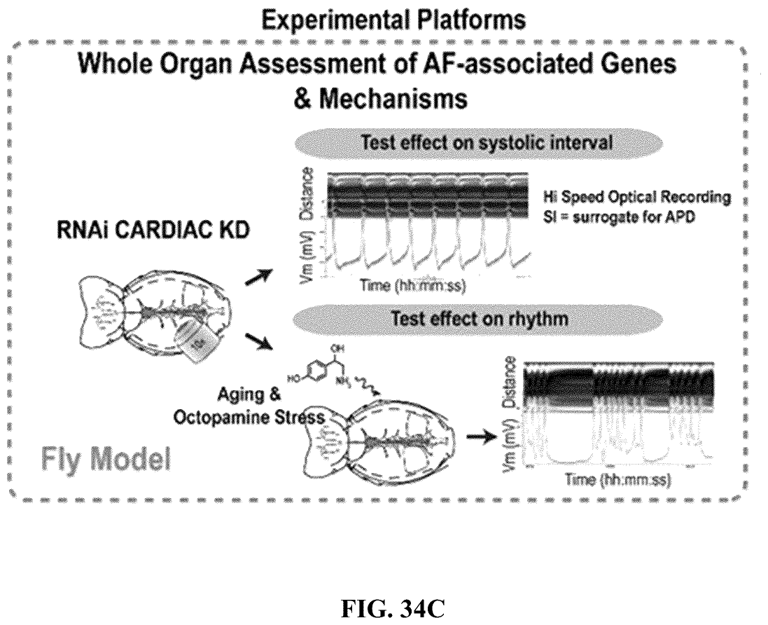

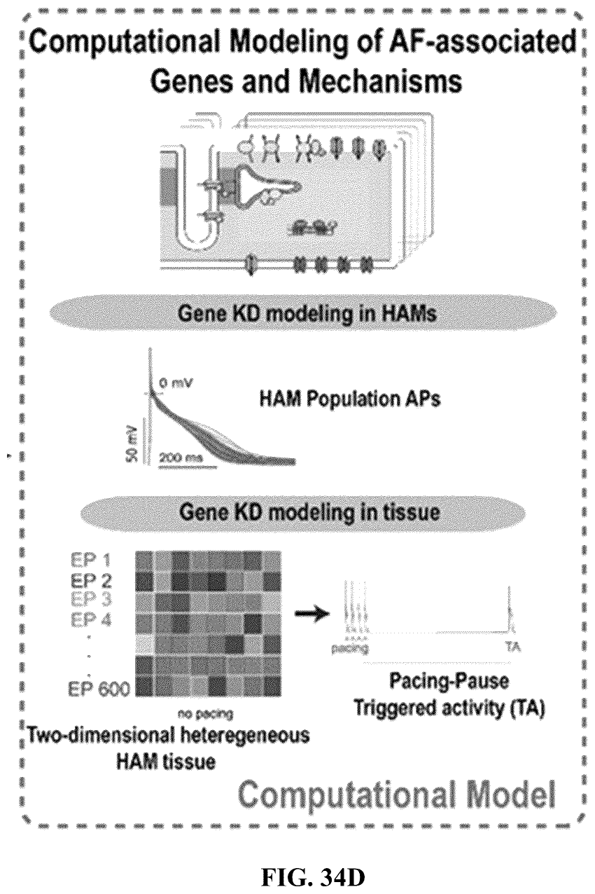

The integrated use of model systems combining functional screening capacity and human atrial and whole organ relevance represents a novel approach enabling the identification and characterization of new genes affecting AF-associated rhythm biology with unprecedented throughput as shown in FIGS. 34A-D.

FIGS. 34A-D shows novel multiplatform modeling of atrial fibrillation. Several schematics are shown summarizing how the integrated multiplatform approach of the present disclosure enables the HT identification and characterization of AF-associated genes and mechanism, using model systems with human, adult, whole organ, and atrial relevance. FIG. 34A shows a schematic showing that AF-associated gene candidates are screened via HT phenotypic characterization. FIG. 34B shows a schematic overview of the use of the ACM model of the present disclosure in the cellular assessment of AF-associated genes and mechanisms. FIG. 34C shows a schematic overview of the use of the fly model of the present disclosure for whole organ assessment of AF-associated genes and mechanisms. FIG. 34D shows a schematic overview of the use of the computational model of the present disclosure in modeling AF-associated genes and mechanisms.

### Example 9: Arrhythmias are Modeled in Human Atria Tissue

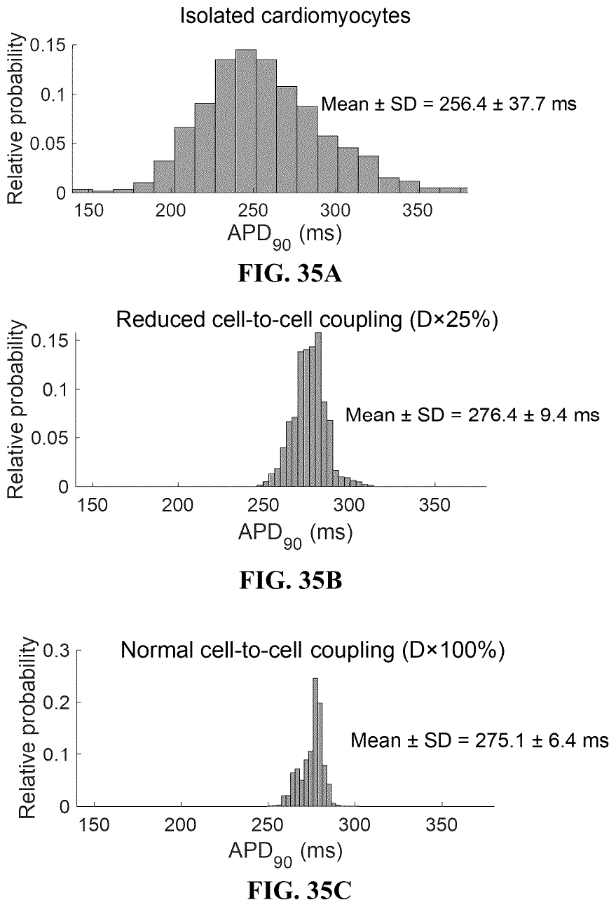

In this example, mathematical modeling is used to assess the behavior of atria tissue. Two-dimensional (2D) models were created to understand the dynamic behaviors of atrial AP and Ca2+ in tissue using a monodomain equation (Clayton et al., 2011) to describe the tissue electrical coupling (see Ni et al., 2017):

$$\frac{\partial {V}_{m}}{\partial t} = D Δ {V}_{m} - \frac{{i}_{ion}}{{C}_{m}}$$

where Vₘ is the membrane potential of cardiomyocytes, iᵢₒₙ represents total ionic current, Cm is the capacitance of the cell membrane, and D indicates the isotropic diffusion coefficient describing the cell-to-cell coupling strength. The 2D model comprises 120×125 grids with a spatial interval of 0.25 mm. To account for the intrinsic variabilities in tissue, the population of 600 models was mapped to the tissue based on a heterogeneous pattern dividing the tissue into 600 blocks consisting of 5×5 grids. Under normal coupling, D=0.1485 mm² ms, so that the conduction velocity under normal conditions is aligned with previous experimental observations and consistent with modeling studies. To assess how tissue coupling affects the arrhythmic events, a reduced coupling (scale to 25% of tissue conductivity) condition was also simulated. The resulting conduction velocity with normal or reduced tissue coupling is given in Table 7. The simulations of the present disclosure showed that the APD variations seen at the single-cell level are reduced in coupled tissue (FIGS. 35A-C), and this is associated with the strength of tissue coupling: increasing the cell-to-cell coupling further reduce the APD variation. FIGS. 35A-C shows normalized histogram plots illustrating distribution of APD90 for the human atrial population of isolated cardiomyocytes (FIG. 35A) and coupled cardiomyocytes in tissue with reduced (FIG. 35B) or normal (FIG. 35C) cell-to-cell coupling. The mean and SD values are indicated in each panel.

### Example 10: Examples of Methodological Approaches Used Herein

ACMs for the study were developed by dissociating Id1 overexpressing hiPSCsl with 0.5 mM EDTA (ThermoFisher Scientific) in PBS without CaCl2 and MgCl2 (Corning) for 7 min at room temperature. hiPSC were resuspended in mTeSR-1 media (StemCell Technologies) supplemented with 2 μM Thiazovivin (StemCell Technologies) and plated in a Matrigel-coated 12-well plate at a density of 3×105 cells per well. After 24 hours after passage, cells were fed daily with mTeSR-1 media (without Thiazovivin) for 3-5 days until they reached ≥90% confluence to begin differentiation. hiPSC-ACMs were differentiated as previously described2. At day 0, cells were treated with 6 μM CHIR99021 (Selleck Chemicals) in S12 media3 for 48 hours. At day 2, cells were treated with 2 μM Wnt-C59 (Selleck Chemicals), an inhibitor of WNT pathway, in S12 media. 48 hours later (at day 4), S12 media is fully changed. At day 5, cells were dissociated with TrypLE Express (Gibco) for 2 min and blocked with RPMI (Gibco) supplemented with 10% FBS (Omega Scientific). Cells were resuspended in S12 media supplemented with 4 mg/L Recombinant Human Insulin (Gibco) (S12+ media), 300 nM retinoic acid (R2625-50 MG) and 2 μM Thiazovivin and plated onto a Matrigel-coated 12-well plate at a density of 9×105 cells per well. S12+ media was changed at day 8 and replaced at day 10 with RPMI (Gibco) media supplemented with 213 μg/μL L-ascorbic acid (Sigma), 500 mg/L BSA-FV (Gibco), 0.5 mM L-carnitine (Sigma) and 8 g/L AlbuMAX Lipid-Rich BSA (Gibco) (CM media). Typically, hiPSC-ACMs start to beat around day 9-10. At day 15, cells were purified with lactate media (RPMI without glucose, 213 μg/μL L-ascorbic acid, 500 mg/L BSA-FV and 8 mM Sodium-DL-Lactate (Sigma)), for 4 days. At day 19, media was replaced with CM media.

### Voltage Assay in ACMs

Voltage assay was performed using the labeling protocol described herein. Briefly, hiPSC-ACMs at day 25 of differentiation were dissociated with TrypLE Select 10× for up to 10 min and the action of TrypLE was neutralized with RPMI supplemented with 10% FBS. Cells were resuspended in RPMI with 2% KOSR (Gibco) and 2% B27 50× with vitamin A (Life Technologies) supplemented with 2 μM Thiazovivin and plated at a density of 6,000 cells per well in a Matrigel-coated 384-well plate. hiPSC-ACMs were then transfected with siRNAs directed against AFib-associated genes (ON-TARGETplus Human, siGATA4: J-008244-05-0002, siGATA5: J-010324-06-0005, siGATA6: J-008351-06-0005, siGJA1: J-011042-05-0002, siGJA5: J-017368-05-0002, siHAND2: J-008698-06-0005, siHCN4: J-006203-05-0002, siKCNA5: J-006215-06-0005, siKCND3: L-006226-00-0005, siKCNJ5: J-006250-06-0002, siKCNN3: J-006270-06-0002, siNKX2-5: J-019795-07-0002, siNKX2-6: J-025793-17-0002, siNPPA: J-012729-05-0002, siPITX2: J-017315-05-0005, siPLN: J-011754-05-0005, siSH3PXD2A: J-006657-07-0002, siSYNE2: J-019259-09-0002, siTBX5: J-013410-5-0002, siZFHX3: J-015410-5-0002) using lipofectamine RNAi Max (ThermoFisher). Each siRNA was tested in biological quadruplicates for each differentiation and differences between experimental conditions and controls were replicated in at least 2 independent differentiations. Every three days post-transfection, cells were first washed with pre-warmed Tyrode's solution (Sigma) by removing 50 μL of media and adding 50 μL of Tyrode's solution 5 times using a 16-channel pipette. After the fifth wash, 50 μL of 2× dye solution consisting in voltage-sensitive dye Vf2.1 Cl (Fluovolt, 1:2000, ThermoFisher) diluted in Tyrode's solution supplemented with 1 μL of 10% Pluronic F127 (diluted in water, ThermoFisher) and 20 μg/mL Hoescht 33258 (diluted in water, ThermoFisher) was added to each well. The plate was placed back in the 37° C. 5% CO2 incubator for 45 min. After incubation time, cells were washed 4 times with fresh pre-warmed Tyrode's solution using the same method described above. hiPSC-ACMs were then automatically imaged with ImageXpress Micro XLS microscope at an acquisition frequency of 100 Hz for a duration of 5 sec with an excitation wavelength of 485/20 nm and emission filter 525/30 nm. A single image of Hoescht was acquired before the time series. Fluorescence over time quantification and trace analysis were quantified using custom software packages. Although, cells were not paced during the APD measurement process, beat rate was controlled in silico by only comparing APDs between conditions where peak trains had similar beat rate (+/−10%), thereby minimizing the effect of beat rate on APD.

### Arrhythmia Assay and Drug Treatment in ACMs

hiPSC-ACMs were dissociated, plated in 384-well plate and transfected with siRNA-associated AFib (ON-TARGETplus Human, siNCX: J-007620-05-0002). 24 hours post-transfection, 2,000 primary human fibroblasts were added per well to the hiPSC-ACMs. 48 hours later (the day of the imaging), cells were dyed with the voltage sensitive dye Vf2.1 Cl as described above, then treated with 50 μL of 2× solution of isoproterenol (1 μM final) diluted in Tyrode alone and in combination with 2× solution of Verapamil (30 nM final), diluted in Tyrode at the 5th wash. After 20 minutes of compound incubation time, cells were imaged, and single-cell traces analyzed as described previously.

### Whole Cell Patch Clamp Electrophysiology

Cardiac ion currents were recorded from single cardiomyocytes using the whole-cell patch-clamp method. Briefly, coverslips with ACMs or VCMs were transferred into electrophysiological perfused recording chamber (RC-25-F, Warner Instruments, Hamden, CT) mounted on the stage of an inverted Olympus microscope. Patch pipettes were pulled from thin-wall borosilicate glass capillaries (CORNING 7740, 1.65 mm) with a P-2000 laser pipette puller (Sutter Instruments, California, USA) and had electrode tip resistances between 1.5 and 5.5 M2 with access resistance of <8M (2 for whole-cell patch recordings. Series resistance and cell capacitance were compensated to between 30 and 60% in some voltage-clamp recordings. For current-clamp recordings, pipettes 1 contained (in mM): K aspartate 76, KCl 20, MgCl 2.5, HEPES 10, NaCl 4, CaCl2 6, K4EGTA 10, K2ATP 5 and Na-GTP 0.1 (pH 7.2; 310 mOsm). All recordings were performed at room temperature in Tyrode's solution. Current response traces were acquired using the Axon 200B amplifier. Currents were digitally sampled at 10 kHz using Digidata 1322A digitizer hardware and pClamp 10.2 software (Molecular Devices, California, USA). For both ACMs and VCMs, n=5.

### Drosophila Strains

The Hand 4.2-Gal4 fly line was used as a heart-specific driver line (Brand and Perrimon, 1993). Virgin Hand-Gal4 females were crossed to male flies from UAS-RNAi lines for each AF gene candidate. UAS-RNAi lines and their respective control lines were acquired from the Bloomington Drosophila Stock Center (BDSC, Indiana, United States of America) and Vienna Drosophila Resource Center (VDRC, Vienna, Austria). For each gene candidate, at least 2 different RNAi lines were used; GD and KK were the genetic background lines for stocks from VDRC lines and ATTP2 and ATTP40 were the genetic background lines for stocks from BDSC).

The PLN (fly ortholog: Sarcolamban A/SclA) sensitized fly line was made by recombining the USA-SclA RNAi with the Hand 4.2-Gal4 heart-specific driver line. Virgin females from the Hand-Gal4 or the SclA-sensitized, Hand-Gal4 driver lines were crossed to males of the desired UAS-RNAi lines. Adult female flies for all crosses were collected upon eclosion and raised at 25° C. on a 12-hour light-dark cycle. Flies were fed a standard yeast-cornmeal diet, with food replaced every other day.

### Drosophila Heart Function Characterization

Cardiac phenotypes of middle-aged (3-weeks old) female flies from each cross were characterized using denervated, semi-intact preparations as previously described in (Ocorr et al., 2009; Vogler and Ocorr, 2009). Briefly, hearts from 20-25 flies were examined for each genotype and age. Adult female flies were exposed to FlyNap, a triethylamine-based anesthetic, for at least one minute until no movement was detected. Hearts were exposed by dissection in room temperature, air bubbled, artificial hemolymph (AHL, Ocorr et al, 2007). High-speed video recordings were filmed with a Hamamatsu EM-CCD camera and using HC Image capture software (Hamamatsu Corp). Heart movements were analyzed using the Semi-automated Optical Heartbeat Analysis (SOHA) software (sohasoftware.com). Movies were recorded at speeds of 140+ fps with pixel resolution ˜ 1 micron/pixel allowing very precise temporal and spatial measurements, including heart period (HP) and rate (1/HP), diastolic and systolic intervals, and fractional shortening/contractility. To quantitate arrhythmia, median absolute deviation (MAD) was first calculated. The median value of the absolute deviations of each heart period (Xi) from the median heart period ((X)) was calculated and then multiplied by a constant (k=1.4826 assuming data is normally distributed).

$$MAD = [ median ( \vert X\_i - {X}^{\sim} \vert ) ] * k$$

To normalize the MAD index (nMAD), the MAD value was divided by the median heart period. Qualitative records of heart wall movements (M-modes/kymographs) were produced by electronically excising a 1 pixel horizontal “slice” from each movie frame and aligning them horizontally providing an edge trace displaying heart wall movements in the X-axis over time along the Y-axis (Ocorr et al., 2009; Ocorr et al., 2007c).

### Octopamine-Challenge Heart Assay

Octopamine (OA) pacing experiments were performed in situ on the semi-intact fly preparation. OA (Sigma-Aldrich #00250) stock solution (10 mM) was freshly prepared by dissolving in water and was further diluted in AHL. A dose-response curve was generated using doses ranging from 0.1 nM OA to 500 nM OA (Supplemental FIG. 5A). The increase in heart rate was maximal at 100 nM OA, which was the dose used for all subsequent pacing experiments (Supplemental FIG. 5B). Following dissection, hearts were first allowed to equilibrate in fresh AHL for 15 min and 30 sec movies of heart function were recorded. Heart function was recorded three times per fly: 1) pre-drug exposure, 2) after a 15-minute exposure to 100 nM OA, and 3) after a 15-minute post drug exposure recovery period. A second set of hearts exposed only to vehicle (AHL) were filmed at the same three 15-minute intervals to serve as time controls.

### Simultaneous Optical and Electrophysiological Recordings

Simultaneous optical and intracellular electrical recordings were performed as previously described in (Ocorr et al., 2017). Briefly, a semi-intact preparation was prepared that was incubated in artificial hemolymph. Optical recordings were done as described above; electrical potentials were recorded using sharp glass electrodes (20±50M (2) filled with 3M KCl and standard intracellular electrophysiological techniques. Data were acquired using an Axon-700B Multiclamp amplifier, signals were digitized using the DIGIDATA 1322A and data were captured and analyzed using PClamp 9.0 and Clampfit 10.0 software respectively (all from Molecular Devices). Data was quantified from representative 30 s recordings where the resting membrane potential had remained stable for at least 30 s. To coordinate the optical and electrical recordings a TTL pulse was sent by the image capture software to the Digitizer. The pulse duration lasted for the entire period of optical recording and was recorded in a separate channel by the PClamp software allowing us to delineate the beginning and the end of the optical recording and directly align it with the electrical record.

### Statistical Analysis

ACMs-Population distribution of control and siRNA-treated hiPSC-ACMs was generated with GraphPad Prism software (2019) using nonlinear regression. Unpaired nonparametric Kolmogorov-Smirnov test was used to compare each treated conditions to control using APD75 of every measured cells. To determine any statistical significance between experimental and control groups, two-sided p-values were calculated with Student's t-test using GraphPad Prism software.

Flies-Data that exhibited a normal distribution (Shapiro-Wilk test) was evaluated for significance using a 1-way ANOVA (for simple comparisons) or a 2-way ANOVA (for multiple manipulations) followed by multiple comparisons post-hoc tests as indicated in figure legends. Data sets that did not show a normal distribution (typically heart period, systolic interval and diastolic interval, and arrhythmia parameters) were analyzed using a nonparametric Wilcoxon Rank Sum test or Kruskal-Wallis test followed by Dunn multiple comparisons post-hoc tests. For acute octopamine stress experiments, repeated measures were used, 2-way ANOVA with a Geisser-Greenhouse correction to address potential lack of sphericity, followed by Sidak's multiple comparisons test. If data did not meet assumptions of normality, the data was log-transformed and repeated measures were repeated, 2-way ANOVA. Statistical analysis and data visualization was completed with GraphPad Prism (v8.0.0; graphpad.com), R (v3.6; r-project.org), and Rstudio (v1.3.959; rstudio.com).

### Computational Modeling Design

The computational model of the present disclosure was employed (Grandi et al., 2011) of human atrial myocytes to simulate human action potential (AP) and Ca2+. PLN regulates the function of sarco/endoplasmic reticulum Ca2+-ATPase (SERCA) function by decreasing the apparent affinity of SERCA for Ca2+ ions (Periasamy et al., 2008; Simmerman and Jones, 1998). Accordingly, the effects of PLN knockdown on SERCA were simulated by various degrees of reduction in the SERCA affinity parameter (Kmf) for cytosolic Ca2+: Kmf was scaled by 75%, 50%, or 25% to cover a wide parameter space of change. These changes were made based on a previous study showing that applying PLN antibody shifted the affinity from 0.8 μM to 0.2 μM (Cantilina et al., 1993).

### Modeling Arrhythmias in Human Atrial Cells

To describe the intrinsic cell-to-cell variabilities in atrial electrophysiology and uncover the uncertainty of the modeling results, a population-based approach was applied (Ni et al., 2018; Sobie, 2009) and populations of 600 human atrial model variants were built by randomly perturbing key model parameters (e.g., the maximum ion channel conductances, rates for membrane transporters, and Ca2+ handling fluxes) by a lognormal distribution (φ=0.2).

### Logistic Regression Analysis of Delayed Afterdepolarizations

Logistic regression analysis was performed (Morotti et al., 2017) to understand the influence of each model parameter on the arrhythmic outcome in human atrial myocytes. For each cell of the population of models, a binary code (yes/no) was applied to describe the presence/absence of delayed afterdepolarizations (DADs). Logistic regression coefficients were obtained using MATLAB (R2019b) scripts (Morotti and Grandi, 2017).

### Pacing-and-Pause Protocol in Single Cell and Tissue Stimulation

A constant pacing-and-pause protocol was applied to evaluate the physiological effects of PLN knockdown. Specifically, single cells were paced at 2 Hz for 290 s prior to a 10-s period of pause without stimulation. In tissue simulations, stimuli were applied at the left side of the 2D tissue at 2 Hz for 10 s, which was followed by a 10-s period without stimulation. AP and Ca2+ traces from the last four stimuli and the non-paced period were recorded for data analysis. Logistic regression analysis was applied to uncover the influence of model parameters on the incidence of arrhythmogenic events.

While preferred embodiments of the present disclosure have been shown and described herein, it will be understood by those skilled in the art that such embodiments are provided by way of example only. Numerous variations, changes, and substitutions can be made without departing from the invention. It should be understood that various alternatives to the embodiments described herein can be employed. It is intended that the following claims define the scope of the invention and that methods and structures within the scope of these claims and their equivalents be covered thereby.

1. An in vitro-generated cardiomyocyte wherein:
a. the in vitro-generated cardiomyocyte is generated from a reprogrammed cell in vitro;
b. the in vitro-generated cardiomyocyte comprises at least one gene associated with a cardiac rhythm disorder having an altered expression status; and
c. the in vitro-generated cardiomyocyte displays a phenotype associated with the cardiac rhythm disorder.

2. The in vitro-generated cardiomyocyte of claim 1, wherein the cardiac rhythm disorder is atrial fibrillation (AF).

3. The in vitro-generated cardiomyocyte of claim 1, wherein the cardiomyocyte is an atrial-like cardiomyocyte (ACM).

4. The in vitro-generated cardiomyocyte of claim 1, wherein the gene associated with the cardiac rhythm disorder is selected from the group consisting of: GATA5, GATA6, PITX2, KCNA5, GATA4, KCNJ5, HCN4, GJA1, TBX5, SYNE2, NKX2-6, SH3PXD2A, KCNN3, NPPA, ZFHX3, NKX2-5, HAND2, GJA5, KCND3, and PLN.

5. The in vitro-generated cardiomyocyte of claim 1, wherein the phenotype associated with the cardiac rhythm disorder is an alteration in a cardiac rhythm parameter.

6. The in vitro-generated cardiomyocyte of claim 5, wherein the cardiac rhythm parameter is selected from the group consisting of: action potential duration (APD), systolic interval, beat rate, beat refractory period, peak-to-peak interval, early afterdepolarization, delayed afterdepolarization, and Arrythmia Index (AI) value.

7. The in vitro-generated cardiomyocyte of claim 5, wherein the phenotype is an AI value greater than 20.

8. The in vitro-generated cardiomyocyte of claim 5, wherein the alteration in the cardiac rhythm parameter is a change in the APD₇₅ value, wherein the APD₇₅ value is APD measured at 75% repolarization, or wherein the alteration in the cardiac rhythm parameter is a change in the APD₉₀ value, wherein APD₉₀ is APD measured at 90% repolarization.

9. (canceled)

10. The in vitro-generated cardiomyocyte of claim 5, wherein the alteration in the cardiac rhythm parameter is a shortening of the APD as compared to a reference cardiomyocyte, the alteration in the cardiac rhythm parameter is an increase in the beat refractory period as compared to a reference cardiomyocyte, the alteration in the cardiac rhythm parameter is an increase in beat rate as compared to a reference cardiomyocyte, the alteration in the cardiac rhythm parameter is a reduction in the systolic interval as compared to a reference cardiomyocyte, or the alteration in the cardiac rhythm parameter is a shortening of the Ca²⁺ transient duration as compared to a reference cardiomyocyte.

11.-14. (canceled)

15. The in vitro-generated cardiomyocyte of claim 1, wherein the altered expression status is overexpression of the at least one gene associated with the cardiac rhythm disorder as compared to the expression level of the gene in a reference cardiomyocyte, or wherein the altered expression status is reduced expression of the at least one gene associated with the cardiac rhythm disorder as compared to the expression level in a reference cardiomyocyte.

16. (canceled)

17. The in vitro-generated cardiomyocyte of claim 1, further comprising a nucleic acid molecule capable of modulating the expression of the at least one gene associated with the cardiac rhythm disorder, wherein the nucleic acid molecule is siRNA.

18. (canceled)

19. The in vitro-generated cardiomyocyte of claim 1, wherein the reprogrammed cell is a cardiac progenitor cell, and the cardiac progenitor cell overexpresses Id1.

20. (canceled)

21. The in vitro-generated cardiomyocyte of claim 1, wherein the reprogrammed cell is an induced pluripotent stem cell (iPSC).

22. The in vitro-generated cardiomyocyte of claim 1, wherein the cardiomyocyte expresses one or more genes selected from the group consisting of: NR2F2, TBX5, ZNF385B, KCNJ3, KCNA5, NPPA, NPPB, EGR1/2 and PDGFRA.

23. A cell population comprising at least two in vitro-generated cardiomyocytes of claim 1, wherein the percentage of the cell population that exhibits an AI value of at least 20 is greater than 20%, 30%, 40%, 50%, 60%, 70% or 80%, and wherein the APD₇₅ value measured using a Kolmogorov-Smirnov scale is at least 0.2, 0.3, 0.4, 0.5, 0.6, 0.7, 0.8, or 0.9 when compared to the APD₇₅ value of a reference cardiomyocyte cell population.

24.-25. (canceled)

26. A cell co-culture model of cardiac fibrosis comprising:
a. the in vitro-generated cardiomyocyte of claim 1; and
b. a fibroblast cell, wherein the ratio of the fibroblast cell to the cardiomyocyte cell is 3:1.

27. (canceled)

28. A cell co-culture model of cardiac arrhythmia comprising:
a. the in vitro-generated cardiomyocyte of claim 1; and
b. a pharmaceutical compound, wherein the pharmaceutical compound is isoprotenerol or dofetilide.

29.-30. (canceled)

31. A method for screening a candidate agent for the treatment of a cardiac rhythm disorder comprising:
a. contacting the cardiomyocyte of claim 1 with the candidate agent; and
b. detecting an effect of the candidate agent on the phenotype associated with the cardiac rhythm disorder.

32.-45. (canceled)

46. A method of determining an increased risk for atrial fibrillation (AF) in a human subject comprising:
a. collecting a biological sample from the human subject; and
b. determining by an assay a level of a gene or gene product associated with AF in the biological sample.

47.-56. (canceled)

57. A method for high-throughput identification of a gene underlying a cardiac rhythm disorder comprising:
a. evaluating the effect of the loss-of-function and gain-of-function of the gene on the in vitro-generated cardiomyocyte of claim 1; or
b. evaluating the effect of the loss-of-function and gain-of-function of the gene on a Drosophila heart; or
c. computational modeling of the effect of knockdown of the gene on a computational model of heterogenous adult human atrial myocytes (HAMs).

58.-64. (canceled)
